(19) United States
(12) Patent Application Publication
(10) Pub. No.: US 2022/0213444 A1
(43) Pub. Date: Jul. 7, 2022

(54) COMPOSITIONS AND METHODS FOR CELLULAR REPROGRAMMING

(71) Applicant: CHILDREN'S HOSPITAL MEDICAL CENTER, Cincinnati, OH (US)

(72) Inventor: Takanori Takebe, Cincinatti, OH (US)

(21) Appl. No.: 17/595,495

(22) PCT Filed: May 29, 2020
(86) PCT No.: PCT/US2020/035387

§ 371 Date: Nov. 17, 2021

Related U.S. Application Data
(60) Provisional application No. 62/855,548, filed on May 31, 2019.

(51) Int. Cl. C12N 5/074; C12N 5/073

(52) U.S. Cl. CPC C12N 5/0696; C12N 2502/02; C12N 5/0603

(57) ABSTRACT

Disclosed herein are compositions and methods for re-programming induced pluripotent stem cells, such as from a primed to naïve state. Further disclosed herein are methods of making said reprogrammed induced pluripotent stem cells by contacting the stem cells with another population of stem cells.

### CROSS-REFERENCE TO RELATED APPLICATIONS

This application claims the benefit of priority of U.S. Provisional Patent Application No. 62/855,548, filed May 31, 2019, which is hereby incorporated by reference in its entirety.

### REFERENCE TO SEQUENCE LISTING

The present application is being filed along with a Sequence Listing in electronic format. The Sequence Listing is provided as a file entitled CHMC63_021WOSeqListing.TXT, which was created and last modified on May 28, 2020, which is 3,318 bytes in size. The information in the electronic Sequence Listing is hereby incorporated by reference in its entirety.

### FIELD OF THE INVENTION

Aspects of the present disclosure relate generally to compositions of reprogrammed induced pluripotent stem cells and methods of making thereof.

### BACKGROUND

There have been remarkable advances in cell fate reprogramming, including methods mediated by transcription factor overexpression, nuclear transfer, and cell fusion. However, the epigenetic and phenotypic status of reprogrammed cells is highly variable, limiting the utility of reprogrammed cells for biomedical applications. For example, clone-dependent variability (or differentiation bias) limits the efficiency of some reprogramming protocols. In addition, some iPSCs are refractory to differentiation. There is a present need for more robust and reproducible strategies to stabilize the variable reprogrammed state for applications in precision medicine, drug screening, and cell therapy.

### SUMMARY

Cell fate reprogramming is an important objective in molecular biology, offering the promise to enable disease modeling, drug discovery, and regenerative medicine. Reprogramming requires significant alternation of gene expression signatures specific to the desired cell types. This has previously been achieved by distinct experimental approaches: nuclear transfer, cell fusion, transcription factor gene transduction, and small molecules. As disclosed herein, by employing an experimental co-culture model, cells, such as human cells, in a primed pluripotent state can be reprogrammed into a naïve-like state in the presence of a naïve pluripotent stem cell population. Importantly, this unique inter-cellular mRNA exchange phenomenon, which accounts for a measurable portion of the recipient transcriptome, does not require manual transfer of conventional reprogramming factors. This process is primarily driven by direct cell contacts, possibly through nanotubes connected to neighboring cells, rather than other indirect mechanisms.

Some aspects of the present disclosure relate to methods of reprogramming cells. In some embodiments, the methods comprise contacting in vitro an acceptor cell with a donor cell. In some embodiments, the contacting causes transfer of one or more intra-cellular components from the donor cell to the acceptor cell. In some embodiments, the acceptor cell is a pluripotent stem cell (PSC). In some embodiments, the acceptor cell is a primed PSC. In some embodiments, the acceptor cell is an induced pluripotent stem cell (iPSC). In some embodiments, the acceptor cell is a primed induced pluripotent stem cell. In some embodiments, the acceptor cell is a mammalian cell. In some embodiments, the acceptor cell is a mouse cell. In some embodiments, the acceptor cell is a human cell. In some embodiments, the acceptor cell is a human induced pluripotent stem cell (hiPSC). In some embodiments, the acceptor cell is a primed hiPSC. In some embodiments, the acceptor cell is an epiblast-derived stem cell (EpiSC). In some embodiments, the acceptor cell is a cell in a primed state. In some embodiments, the acceptor cell expresses primed transcription factors and/or primed cell surface markers. In some embodiments, but without being limited by any mechanism of action, the donor cell comprises tunneling nanotubes (TNTs) or cytonemes on its surface. In some embodiments, the donor cell is a PSC. In some embodiments, the donor cell is an iPSC. In some embodiments, the donor cell is a mammalian cell. In some embodiments, the donor cell is a mouse cell. In some embodiments, the donor cell is a human cell. In some embodiments, the donor cell is an hiPSC. In some embodiments, the donor cell is a naïve PSC. In some embodiments, the donor cell is a naïve iPSC. In some embodiments, the donor cell is a naïve hiPSC. In some embodiments, the donor cell is an embryonic stem cell. In some embodiments, the donor cell is a mouse embryonic stem cell (mESC). In some embodiments, the donor cell is a naïve mESC. In some embodiments, the donor cell is a mouse EpiSC. In some embodiments, the donor cell is a cell in a naïve state. In some embodiments, the donor cell expresses naïve transcription factors and/or naïve cell surface markers. In some embodiments, the donor cell and acceptor cell are contacted in a culture media. In some embodiments, the donor cell and acceptor cell are cultured in naïve maintenance medium. In some embodiments, the culture media is RSet medium, naïve human stem cell (NHSM), 5i medium, 4i medium, 3i medium, feeder-independent naïve embryonic (FINE) medium, mTeSR. medium, mTeSR medium with Matrigel, PXGL medium, N2B27 medium, N2 medium, T2iLGö, tt2iLGö medium, 2i medium, or 2i medium with gelatin. In some embodiments, the culture media comprises one or more of a GSK3 inhibitor, MAPK inhibitor, LIF, JNK inhibitor, p38 inhibitor, bFGF, or TGF-β (e.g. at least 1, 3, 5). In some embodiments, the donor cell and acceptor cell are not cultured with a feeder cell. In some embodiments, the donor cell and acceptor cell are cultured with a feeder cell. In some embodiments, the donor cell and the acceptor cell are not contacted or cultured with an HDAC inhibitor. In some embodiments, the donor cell and acceptor cell are cultured at a ratio that is, is about, is at least, is at least about, is not more than, or is not more than about, 1%:99%, 5%:95%, 10%:90%, 15%:85%, 20%:80%, 25%:75%, 30%:70%, 35%:65%, 40%:60%, 45%:55%, 50%:50%, 55%:45%, 60%:40%, 65%:35%, 70%:30%, 75%:25%, 80%:20%, 85%:15%, 90%:10%, 95%:5%, or 99%:1%, or any ratio within a range defined by any two of the aforementioned ratios, for example 1%:99% to 99%:1%, 10%:90% to 90%:10%, 20%:80% to 80%:20%, 30%:70% to 70%:30%, 40%:60% to 60%:40%, 45%:55% to 55%:45%, 1%:99% to 50%:50% or 50%:50% to 99%:1%. In some embodiments, the donor cell and acceptor cell are cultured at a ratio that is, is about, is at least, is at least about, is not more than, or is not more than about, 20%:80%, 40%:60%, 45%:55%, 50%:50%, 55%:45%, 60%:40%, or 80%:20%. In some embodiments, the donor cell and acceptor cell are cultured at a ratio that is, is about, is at leak, is at least about, is not more than, or is not more than about, 50%:50% (1:1). In some embodiments, the intra-cellular component that gets transferred comprises RNA, DNA, nucleic acids, proteins, polypeptides, peptides, or organelles, or any combination thereof. In some embodiments, the intra-cellular component that gets transferred is selected from one or more of RNA, DNA, nucleic acids, proteins, polypeptides, peptides, and organelles (e.g. at least 1, 3, 5). In some embodiments, the intra-cellular component is RNA. In some embodiments, the intra-cellular component is mRNA, ncRNA, lncRNA, miRNA, piRNA, siRNA, or shRNA, or any combination thereof. In some embodiments, but without being limited by any mechanism of action, the donor cell transfers the intra-cellular component via tunneling nanotubes or cytonemes. In some embodiments, after contacting, the acceptor cell comprises exogenous mRNA from the donor cell that is xenogeneic. In some embodiments, after contacting, the acceptor cell comprises exogenous mRNA from the donor cell that is allogeneic or autologous. In some embodiments, after contacting, the acceptor cell comprises exogenous mRNA encoding for a number of genes that is, is about, is at least, is at least about, is not more than, or is not more than about, 100, 200, 300, 400, 500, 1000, 2000, 3000, 4000, 5000, 6000, 6382, 7000, 8000, 9000 genes, or a range defined by any two of the preceding values, for example 100-9000, 2000-8000, 4000-8000, 100-500, or 300-2000 genes. In some embodiments, the genes that the acceptor cell comprises are naïve transcription factors. In some embodiments, the acceptor cell is not modified by transfection, electroporation, or transduction with a virus, or any combination thereof, prior to contacting. In some embodiments, the donor cell and acceptor cell are contacted under hypoxic conditions. In some embodiments, the hypoxic condition comprises, consists essentially or, or consists of a percentage of O₂ that is, is about, is at least, is at least about, is not more than, or is not more than about, 0%, 1%, 2%, 3%, 4%, 5%, 6%, 7%, 8%, 9%, 10%, 11%, 12%, 13%, 14%, 15%, 16%, 17%, 18%, 19%, or 20% O₂, or any concentration of O₂ within a range defined by any two of the aforementioned concentration, for example 0% to 20%, 3% to 10%, 4% to 6%, 0% to 5%, or 5% to 20%. In some embodiments, the hypoxic condition comprises, consists essentially of, or consists of a percentage of O₂ that is, is about, is at least, is at least about, is not more than, or is not more than about, 3%, 4%, 5%, 6%, or 7% O₂. In some embodiments, the hypoxic condition comprises, consists essentially of, or consists of a percentage of O₂ that is, is about, is at least, is at least about, is not more than, or is not more than about, 5% O₂. In some embodiments, the donor cell and acceptor cell are contacted under a stressor condition. In some embodiments, the stressor condition comprises, consists essentially of, or consists of contact with a cell-toxic compound, hypoxia, non-physiological temperature, non-physiological pH, electroporation, or any combination thereof. In some embodiments, the donor cell and acceptor cell are contacted or grown at a temperature that is, is about, is at least, is at least about, is not more than, or is not more than about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C. 49° C. 50° C. or any temperature within a range defined by any two of the aforementioned temperatures, for example, 15° C. to 50° C., 20° C. to 45° C., 25° C. to 40° C., 32° C. to 42° C., or 35° C. to 39° C. In some embodiments, the donor cell and acceptor cell are contacted or grown at a temperature that is, is about, is at least, is at least about, is not more than, or is not more than about, 37° C. In some embodiments, the donor cell and acceptor cell are grown with direct contact of the acceptor cell and donor cell. In some embodiments, the donor cell and acceptor cell are directly contacted. In some embodiments, the donor cell and acceptor cell are cultured in direct contact. In some embodiments, the donor cell and acceptor cell are not separated with a transwell. In some embodiments, the donor cell and acceptor cell are not contacted in the presence of donor cell-conditioned medium. In some embodiments, the contacting step is carried out until the acceptor cell expresses or upregulates expression of one or more naïve stem cell markers (e.g. CD130, CD77, CD7, CD75, or F11R). In some embodiments, contacting step is carried out until the acceptor cell exhibits downregulation of one or more primed stem cell markers (e.g. CD90, HLA-ABC, CD24, CD57, or SSEA4). In some embodiments, the contacting step results in increased expression of naïve pluripotency markers or transcription factors (e.g. KLF4, KLF5, KLF17, TFCP2L1, DNMT3L, DPPA3, DPPA5, PRDM14, SALL4, ESRRB, TFAP2C, or TBS3) and downregulation of primed pluripotency markers or transcription factors (e.g. ZIC2, ZIC3, OTX2, DUSP6, THY1, FOXA2, or XIST) in the acceptor cell. In some embodiments, the contacting step is carried out until dome-shaped naïve acceptor colonies are observed. In some embodiments, after contacting, the acceptor cell undergoes changes in chromatin accessibility. In some embodiments, the changes in chromatin accessibility comprises increased accessibility to binding motifs for SOX2 or TFAP2C, or both. In some embodiments, one or both of the donor cell or acceptor cell are contacted with at least one agent selected from the group consisting of resveratrol, epigallocatechin gallate (EGCG), curcumin, genistein, activin-A, Wnt-3a, sodium butylate, basic fibroblast growth factor (bFGF), oncostatin M (OSM), dexamethasone (DEX), hepatocyte growth factor (HGF), CHIR-99021, forskolin, Y-27632. (ROCK inhibitor), (s)-(−)-blebbistatin, IWP2, A83-01, LY294002, SB-431542, NVP-BHG, Cyclopamine-KAAD, PD-0325901, FGF4, LDN-193189, insulin like growth factor (IGF), bone morphogenetic protein 2 (BMP2), transforming growth factor β2 (TGF-β2), BMP4, FGF-7, platelet-derived growth factor (PDGF) β3, epidermal growth factor (EGF), exendin-4, human neuregulin (hEIRG) β3, retinoic acid (RA), L-Ascorbic acid 2-phosphate (AA2P), ascorbic acid, insulin-transferrin-selenium ethanolamine solution (ITS-X), insulin, rifampicin, penicillin, streptomycin, 2-mercaptoethanol, 3-mercaptopropane-1,2-diol (thioglycerol), L-proline, L-glutamine, non-essential amino acid mixture (NEAA), sodium pyruvate, trypsin-EDTA, phosphatidylinositol (PI), interleukins, prostaglandins, and tumor necrosis factors, or any combination thereof. In some embodiments, one or both of the donor cell or acceptor cell are contacted with at least one agent selected from the group consisting of GSK3 inhibitors (e.g. CHIR99021), MAPK inhibitors (e.g. PD0325901), LIF, JNK inhibitors (e.g. SP600125), p38 inhibitors (e.g. SB203580), ROCK inhibitors (e.g. Y-27632), PKC inhibitors (e.g. Gö 6983), BMP inhibitors (e.g. dorsomorphin), bFGF, Activin A, ascorbic acid, cAMP activator (e.g. forskolin), or TGF-β inhibitors (e.g. A83-01), or any combination thereof. In some embodiments, the donor cell and acceptor cell are contacted or cultured for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days. In some embodiments, the donor cell and acceptor cell are contacted or cultured for a number of days that is, is about, is at least, is at least about, is not more than, or is not more than about, 1, 2, 3, 4, 5, 6, 7, 9, or 10 days.

Some aspects of the present disclosure relate to cell compositions. In some embodiments, the cell compositions comprise an acceptor cell and a donor cell. In some embodiments, the acceptor cell is a PSC. In some embodiments, the acceptor cell is a primed PSC. In some embodiments, the acceptor cell is an iPSC. In some embodiments, the acceptor cell is a primed induced pluripotent stem cell. In some embodiments, the acceptor cell is a mammalian cell. In some embodiments, the acceptor cell is a mouse cell. In some embodiments, the acceptor cell is a human cell. In some embodiments, the acceptor cell is a hiPSC. In some embodiments, the acceptor cell is a primed hiPSC. In some embodiments, the acceptor cell is an EpiSC. In some embodiments, the acceptor cell is a naïve pluripotent stem cell. In some embodiments, the acceptor cell is a human naïve pluripotent stem cell. In some embodiments, the acceptor cell is human naïve iPSC. In some embodiments, but without being limited by any mechanism of action, the donor cell is any cell that has tunneling nanotubes. In some embodiments, the donor cell is a PSC. In some embodiments, the donor cell is an iPSC. In some embodiments, the donor cell is a mammalian cell. In some embodiments, the donor cell is a mouse cell. In some embodiments, the donor cell is a human cell. In some embodiments, the donor cell is an hiPSC. In some embodiments, the donor cell is a naïve PSC. In some embodiments, the donor cell is a naïve iPSC. In some embodiments, the donor cell is a naïve hiPSC. In some embodiments, the donor cell is an embryonic stem cell. In some embodiments, the donor cell is an mESC. In some embodiments, the donor cell is a naïve mESC. In some embodiments, the donor cell is a mouse EpiSC. In some embodiments, the acceptor cell is a pluripotent stem cell, and the donor cell is a naïve pluripotent stem cell. In some embodiments, the cell composition further comprises a naïve maintenance medium. In some embodiments, the naïve maintenance medium is mTeSR medium, mTeSR medium with Matrigel, PXGL, medium, N2B27 medium, N2 medium, tt2iLGö medium. 2i medium, or 2i medium with gelatin. In some embodiments, the cell composition does not comprise a feeder cell. In some embodiments, the cell composition comprises a feeder cell. In some embodiments, the donor cell and acceptor cell are at a ratio that is, is about, is at least, is at least about, is not more than, or is not more than about, 1%:99% 5%:95%, 10%:90%, 15%:85%, 20%:80%, 25%:75%, 30%:70%, 35%:65%, 40%:60%, 45%:55%, 50%:50%, 55%:45%, 60%:40%, 65%:35%, 70%:30%, 75%:25%, 80%:20%, 85%:15%, 90%:10%, 95%:5%, or 99%:I%, or any ratio within a range defined by any two of the aforementioned ratios, for example 1%:99% to 99%:1%, 10%:90% to 90%:10%, 20%:80% to 80%:20%, 30%:70% to 70%:30%, 40%:60% to 60%:40%, 45%:55% to 55%:45%, 1%:99% to 50%:50% or 50%:50% to 99%:1%. In some embodiments, the donor cell and acceptor cell are at a ratio that is, is about, is at least, is at least about, is not more than, or is not more than about, 20%:80%, 40%:60%, 45%:55%, 50%:50%, 55%:45%, 60%:40%, or 80%:20%. In some embodiments, the donor cell and acceptor cell are at a ratio that is, is about, is at least, is at least about, is not more than, or is not more than about, 50%:50% (1:1). In some embodiments, the acceptor cell comprises exogenous mRNA from the donor cell that is xenogeneic. In some embodiments, the acceptor cell comprises exogenous mRNA from the donor cell that is allogeneic or autologous. In some embodiments, the acceptor cell comprises exogenous mRNA encoding for a number of genes that is, is about, is at least, is at least about, is not more than, or is not more than about, 100, 200, 300, 400, 500, 1000, 2000, 3000, 4000, 5000, 6000, 6382, 7000, 8000, 9000 genes, or a range defined by any two of the preceding values, for example 100-9000, 2000-8000, 4000-8000, 100-500, or 300-2000 genes. In some embodiments, the genes that the acceptor cell comprises are naïve transcription factors. In some embodiments, the acceptor cell is not modified by transfection, electroporation, or transduction with a virus, or any combination thereof. In some embodiments, the acceptor cell expresses or upregulates expression of one or more naïve stem cell markers (e.g. CD130, CD77, CD7, CD75, or F11R). In some embodiments, the acceptor cell exhibits downregulation of one or more primed stem cell markers (e.g. CD90, HLA-ABC, CD24, CD57, or SSEA4). In some embodiments, the acceptor cell expresses or upregulates expression of one or more naïve pluripotency markers or transcription factors (e.g. KLF4, KLF5, KLF17, TFCP2L1, DNMT3L, DPPA3, DPPA5, PRDM14, SALL4, ESRRB, TFAP2C, or TBS3). In some embodiments, the acceptor cell downregulates expression of one or more primed pluripotency markers or transcription factors (e.g. ZIC2, ZIC3, OTX2, DUSP6, THY1, FOXA2, or XIST). In some embodiments, the cell composition further comprises at least one agent selected from the group consisting of resveratrol, epigallocatechin gallate (EGCG), curcumin, genistein, activin-A, Wnt-3a, sodium butylate, basic fibroblast growth factor (bFGF), oncostatin M (OSM), dexamethasone (DEX), hepatocyte growth factor (HGF), CHIR-99021, forskolin, Y-27632 (ROCK inhibitor), (s)-(−)-blebbistatin, IWP2, A83-01, LY294002, SB-431542, NVP-BHG, Cyclopamine-KAAD, PD-0325901, FGF4, LDN-193189, insulin like growth factor (IGF), bone morphogenetic protein 2 (BMP2), transforming growth factor β2 (TGF-β2), BMP4, FGF-7, platelet-derived growth factor (PDGF) β3, epidermal growth factor (EGF), exendin-4, human neuregulin (hEIRG) β3, retinoic acid (RA), L-Ascorbic acid 2-phosphate (AA2P), ascorbic acid, insulin-transferrin-selenium ethanolamine solution (ITS -X), insulin, rifampicin, penicillin, streptomycin, 2-mercaptoethanol, 3-mercaptopropane-1,2-diol (thioglycerol), L-proline, 1-glutamine, non-essential amino acid mixture (NEAA), sodium pyruvate, trypsin-EDTA, phosphatidylinositol (PI), interleukins, prostaglandins, and tumor necrosis factors, or any combination thereof. In some embodiments, the cell composition further comprises at least one agent selected from the group consisting of GSK3 inhibitors (e.g. CHIR99021), MAPK inhibitors (e.g. PD0325901), JNK inhibitors (e.g. SP600125), p38 inhibitors (e.g. SB203580), ROCK inhibitors (e.g. Y-27632), PKC inhibitors (e.g. Gö 6983), BMP inhibitors (e.g. dorsomorphin), bFGF, Activin A, ascorbic acid, cAMP activator (e.g. forskolin), or TGF-β inhibitors (e.g. A83-01), or any combination thereof.

Embodiments of the present invention provided herein are described by way of the following numbered alternatives:

1. A method comprising contacting an acceptor cell with a donor cell, wherein said contacting causes transfer of an intra-cellular component from said donor cell to said acceptor cell.

2. The method of alternative 1, wherein said acceptor cell is a pluripotent stem cell (PSC),

3. The method of alternative 1 or 2, wherein said acceptor cell is a primed human induced pluripotent stem cell (“primed hiPSC”).

4. The method of any preceding alternative, wherein said donor cell is any cell that has nanotubes.

5. The method of any preceding alternative, wherein said donor cell is a cell in a naïve state expressing naïve transcription factors.

6. The method of any preceding alternative, wherein said donor cell is a naïve mouse embryonic stem cell (naïve mESC).

7. The method of any preceding alternative, wherein said donor cell and said acceptor cell are contacted in a culture media.

8. The method of any preceding alternative, wherein said donor cell and said acceptor cell are cultured at a 1:1 ratio.

9. The method of any preceding alternative, wherein said intra-cellular component is selected from one or more of RNA, protein, and organelles.

10. The method of any preceding alternative, wherein said intra-cellular component is RNA.

11. The method of any preceding alternative, wherein said donor cell transfers the intra-cellular component via tunneling nanotubes or cytonemes.

12. The method of any preceding alternative, wherein the donor cell and acceptor cell are contacted under hypoxic conditions.

13. The method of alternative 12, wherein said hypoxic condition is about 5% O₂.

14. The method of any of alternatives 1 through 11, wherein the donor cell and acceptor cell are contacted under a stressor condition, wherein said stressor condition is selected from contact with a cell-toxic compound, hypoxia, non-physiological temperature, non-physiological pH, electroporation, or any combination thereof.

15. The method of any of alternatives 1 through 11, wherein said cells are contacted or grown at about 37° C.

16. The method of any preceding alternative, wherein said acceptor cell and said donor cell are grown with direct contact of said acceptor cell and said donor cell.

17. The method of any preceding alternative, wherein said contacting step is carried out until said acceptor cell expresses naïve stem cell markers (CD130, CD77).

18. The method of any preceding alternative, wherein said contacting step is carried out until said acceptor cell exhibits downregulation of primed stem cell markers (CD90, HLA-ABC).

19. The method of any preceding alternative, wherein said contacting step results in increased expression of naïve pluripotency markers (DPPA3, TFCP2L1, DNMT3L, KLF4 and KLF17), and downregulation of primed pluripotency markers (DUSP6, THY1) in said acceptor cell.

20. The method of any preceding alternative, wherein said contacting step results in expression of naïve markers KLF17 and TFAP2C in said acceptor cell.

21. The method of any preceding alternative, wherein said contacting step is carried out until dome-shaped naïve acceptor colonies are observed.

22. The method of any preceding alternative comprising contacting one or both of said donor or acceptor cell with at least one agent selected from the group consisting of resveratrol, epigallocatechin gallate (EGCG), curcumin, genistein, activin-A, Wnt-3a, sodium butylate, basic fibroblast growth factor (bFGF), oncostatin M (OSI), dexamethasone (DEX), hepatocyte growth factor (HGF), CHIR-99021, forskolin, Y-27632 (ROCK inhibitor), (s)-(−)-blebbistatin, IWP2, A83-01, LY294002, SB-431542, NVP-BHG, Cyclopamine-KAAD, PD-0325901, FGF4, LDN-193189, insulin like growth factor (IGF), bone morphogenetic protein 2 (BMP2), transforming growth factor β2 (TGF-β2), BMP4, FGF-7, platelet-derived growth factor (PDGF) β3, epidermal growth factor (EGF), exendin-4, human neuregulin (hHRG) β3, retinoic acid (RA), L-Ascorbic acid 2-phosphate (AA2P), ascorbic acid, insulin-transferrin-selenium ethanolamine solution (ITS-X), insulin, rifampicin, penicillin, streptomycin, 2-mercaptoethanol, 3-mercaptopropane-1,2-diol (thioglycerol), L-proline, L-glutamine non-essential amino acid mixture (NEAA), sodium pyruvate, trypsin-EDTA, phosphatidylinositol (PI), interleukins, prostaglandins, and tumor necrosis factors, or any combination thereof.

### BRIEF DESCRIPTION OF THE DRAWINGS

In addition to the features described above, additional features and variations will be readily apparent from the following descriptions of the drawings and exemplary embodiments. It is to be understood that these drawings depict embodiments and are not intended to be limiting in scope.

FIG. 1A depicts an overview of an embodiment of an experimental design for human iPSC (hiPSC) expressing EGFP co-culture with mouse feeder (mFeeder) SNL cells. Human and mouse cell fractions were sorted by flow cytometry for downstream analysis. Bar, 200 μm.

FIG. 1B depicts an embodiment of RT-PCR analysis of human/mouse specific ACTB/Actb and NEAT1/Neat1 expression levels in sorted hiPSC and SNL 76/7 mouse feeder (mFeeder) fractions after 5 days of co-culture. NEAT1Neat1, which is located in cell nuclei and therefore cannot be transferred between cells, is shown as a negative control.

FIG. 1C depicts an embodiment of RT-PCR analysis of human/mouse specific ACTB/Actb expression levels in sorted hiPSC co-cultured with SNL 76/7 mouse feeder cells vs. hiPSC cultured alone or SNL 76/7 mouse feeder cells as well as sorted mouse SNL cells co-cultured with hiPSCs vs. hiPSC or SNL cells cultured alone for 5 days using hiPSC cell lines TkDA3-4-GFP, 1383D6-GFP, 317D6-GFP, and FF-101-GFP. After co-culture conditions, hiPSCs exhibited mouse Actb mRNA, and mouse SNL cells exhibited human ACTB mRNA.

FIG. 1D depicts an embodiment of hiPSCs cultured either alone (upper panels) with mFeeder cells (lower panels) observed by SEM. The hiPSCs cultured with SNL cells show protruding nanotube structures extending between the two cell types.

FIG. 1E depicts an embodiment of mESCs cultured with mFeeder cells with visible nanotube structures (upper panels) and impairment of nanotube formation by LPS treatment between mESCs and mFeeder cells (lower panels) as observed by SEM. Dotted lines indicate the cell-cell interface. The bidirectional arrows indicate the inter-cellular gap.

FIG. 1F depict an embodiment of nanotube structures between mFeeder and hiPSCs observed by SEM (upper panels) that was impaired by LPS treatment (lower panels). Dotted lines indicate the cell-cell interface.

FIG. 1G depicts an embodiment of quantitative RT-PCR analysis of endogenous and transferred human/mouse specific ACTB/Actb in FIG. 1F.

FIG. 2A depicts an overview of an embodiment of experimental design for hiPSC co-culture with mouse embryonic stem cells (mESC) followed by serial sorting of hiPSC.

FIG. 2B depicts an embodiment of quantitative RT-PCR analysis of mouse specific Actb and Nanog expression level in sorted hiPSCs after co-culturing with mESCs for 5 days (upper graph). The values indicate the detected levels in hiPSCs relative to endogenous expression in mESCs. FIG. 2B also depicts quantitative RT-PCR analysis of human specific ACTB and NANOG expression level in sorted mESC after co-culturing with hiPSCs (lower graph). The values indicate the detected levels in mESCs relative to endogenous expression in hiPSCs.

FIG. 2C depicts an embodiment of immunofluorescence micrographs of the morphological change in three hiPSC clones (317-12-EGFP, 317D6-EGFP, TKDA-mCherry) co-cultured with mESC or mESC-OCT4-EGFP at day 5.

FIG. 2D depicts an embodiment of a heatmap of differentially expressed mouse genes in hiPSCs and gene ontology (GO) categories of the genes. The top 75 genes with the highest gene expression variance among the samples were plotted in the heatmap. An increase in detected mouse genes is seen for all 4 tested cell lines.

FIG. 2E depicts an embodiment of selected naïve and primed pluripotent related mRNAs in hiPSCs after co-culturing with mESCs by RNA-seq.

FIG. 3A depicts an embodiment of proliferation and morphology change in mESC, hiPSC, and hiPSC mixed with mESC from day 1 to day 5. Light gray signal in right panels correspond to 317D6-EGFP hiPSC cells.

FIG. 3B depicts an embodiment of quantitative RT-PCR analysis of primed-state related (DUSP6) and naïve-state related (DPPA3, DNMT3L, TFCP2L1, KLF4, KTF17) genes expression in hiPSCs after the co-culture with mESCs. The graphs show the fold-change in sorted hiPSCs experiencing the co-culture with mESCs versus hiPSCs only from three independent experiments.

FIG. 3C depicts an embodiment of quantitative RT-PCR analysis of naïve-state related (DPPA3, DNMT3L, TECP2L1, KLF4, KLF17) genes expression in hiPSCs after culture in mESC-conditioned media or a transwell co-culture assay with mESCs (upper graphs). The graphs show the fold-change in these hiPSCs compared to sorted hiPSCs cultured with mESCs. FIG. 3C also depicts quantitative RT-PCR analysis of mouse Actb in hiPSCs after either co-culture with mESCs, in a transwell co-culture assay with mESCs, or culture in mESC-conditioned media.

FIG. 3D depicts an embodiment of flow cytometric analysis of primed-specific markers (CD90 and HLAABC) and naïve-specific markers (CD130 and CD77) in hiPSC culture either alone or with mESC.

FIG. 3E depicts an embodiment of immunostaining of human naïve markers KLF4, CP2L1 or human nuclear antigen (HuNu) in parental hiPSC, sorted and proliferated hiPSC after co-culture with mESC, or hiPSC after chemical resetting methods.

FIG. 3F depicts an embodiment of principal component analysis (PCA) based on genes differentially expressed between naïve and conventional PSC for chemical reset cells (CR), mixed cultured cells with mESCs (Mixed), those parental iPSCs (Primed), deposit dataset of Shef6 primed ESC (Shef6-primed), and its chemical reset cells (Shef6-cR) from RNA-seq datasets. PCI explains 57% and PC2 explains 18% of analyzed gene set. Each set has three dots corresponding to three replicates.

FIG. 3G depicts an embodiment of hierarchical clustering of the datasets obtained by RNA-seq.

FIG. 3H depicts an embodiment of averaged normalized RNA-seq counts of naïve and primed markers in the cells of FIGS. 3E-G.

FIG. 3I depicts an embodiment of quantitative RT-PCR analysis of human primed (DUSP6) and naïve (DPPA3, DNMT3L, TFCP2L1, KLF4, KLF17) genes expression in different hiPSC cell lines (317-12, 317D6, TKDA) co-cultured with mESCs.

FIG. 3J depicts an embodiment of quantitative RT-PCR analysis of human primed (DUSP6) and naïve (DPPA3, DNMT3L, TFCP2L1, KLF4, KLF17) genes expression in hiPSCs co-cultured with mESCs at different ratios (2:8, 5:5, 8:2, 10:0). The hiPSCs showed greater naïve marker gene expressed with greater the relative number of mESCs.

FIG. 3K depicts SEM analysis of an embodiment of hiPSCs co-cultured with mESCs showing the loss of nanotubes in the presence of LPS and transfer of mouse Actb and Nanog in the hiPSCs. LPS low: 100 ng/mL; LPS high: 500 ng/mL.

FIG. 3L depicts an embodiment of morphology of hiPSC (expressing mCherry) co-cultured with mESC (expressing GFP) under LPS treatment.

FIG. 3M depicts an embodiment of quantitative RT-PCR analysis of human primed (DUSP6) and naïve (DPPA3, DNMT3L, TFCP2L1, KLF4, KLF17) genes expression in hiPSCs experiencing co-culture with mESCs in the presence or absence of LPS treatment. LPS low: 100 ng/mL; LPS high: 500 ng/mL.

FIG. 4A depicts an embodiment of ATAC-seq analysis of hiPSCs before and after co-culture with mESCs. One culture using 317-12 and two replicate cultures using 317-D6 were analyzed.

FIG. 4B depicts an embodiment of a global view of chromatin accessibility changes in hiPSCs before and after co-culture with mESCs. ATAC-seq was performed in three separate experiments. ATAC-seq peaks were identified and classified into those shared between the two conditions (top), those that closed upon co-culturing (middle) and those that opened upon co-culturing (bottom).

FIG. 4C depicts an embodiment of transcription factor (TF) binding site motif enrichment analysis in hiPSC 317-D6 line ATAC-seq peaks (replicate 1) with or without mESC co-culture. Each point represents a TF binding motif. The X-axis indicates motif enrichment in “co-culture lost” peaks. The Y-axis indicates enrichment in “co-culture gained” peaks.

FIG. 4D depicts an embodiment of similar TF binding site motif enrichment analyses as shown in FIG. 4C for 317-D6 (replicate 2) and 317-12 hiPSC lines.

FIG. 4E depicts an embodiment of a screenshot of the UCSC genome browser depicting the promoter region of the human TFAP2C gene. ATAC-seq signal in hiPSCs with or with co-culture with mESC cells is shown.

FIG. 4F depicts an embodiment of a schematic representation of mRNA transfer-induced naïve-like conversion in human pluripotent stem cells co-cultured with mESCs, a′: transfer of TF-encoding mRNAs occurs between neighboring primed hiPSC and naïve mESC upon co-culture; b′: chromatin reorganization and epigenetic modification at the corresponding IF binding loci are induced during the conversion into naïve-like state; c′: the primed hiPSC is reprogrammed into an early naïve-state like cell.

FIG. 5A depicts an embodiment of shRNAs used herein targeting mouse Klf4, Tfcp2l1, and Tfap2c. Sequence comparison of the targeting sequence of each shRNA with the human orthologue is shown. The number of mismatches between the mouse targeting sequences and human orthologue is shown.

FIG. 5B depicts an embodiment of the validation of shRNA knockdown efficiency in mouse ESC, Quantitative RT-PCR analysis of Klf4, Tfcl2l1, Tfap2c, Pou5f1, and Nanog from mouse ESC infected with Luciferase- (shLuc) or mouse transcription factor-targeting shRNA. Data are expressed as fold of the value of shLuc. Values are shown as mean±SEM (n=3). Differences were analyzed by ANOVA and the Tukey post hoc test. **P<0.01, ***P<0001, and ****P<0.0001 versus shLuc.

FIG. 5C depicts an embodiment of quantitative RT-PCR analysis of KLF4, TFCP2L1, TfAP2C, POU5F1, and NANOG from human iPSC infected with Luciferase- (shLuc) or mouse transcription factor-targeting shRNAs. Data are expressed as fold of the value of shLuc. Values are shown as mean±SEM (n=3).

FIG. 5D depicts an embodiment of an overview of experimental design for human iPSC expressing mouse transcription factor targeting shRNAs (puromycin resistant) co-culture with mouse ESC (puromycin sensitive) followed by puromycin selection and expansion of human iPSC in PGXL medium.

FIG. 5SE depicts an embodiment of the formation of proliferating an iPSC colonies expressing mouse transcription factor targeting shRNAs after puromycin selection. Bar, 100 μm.

FIG. 5F depicts an embodiment of quantification of domed shape colony number in FIG. 5E. The colonies were counted from at least 15 different observation fields.

FIG. 5G depicts an embodiment of bright-field (left) and immunostaining (right) images for hiPSCs before and after mouse ESC co-culture followed by puromycin selection. Bars, 100 μm (bright-field) or 10 μm (immunostaining).

FIG. 6A depicts an embodiment of immunostaining of pan-Oct4 (clone: C30A3) and mouse-specific Oct4 (clone: D6C8T) antibodies for human iPSC and mouse ESC.

FIG. 6B depicts an embodiment of immunostaining of human-specific Nanog (clone: D73G4) and mouse-specific Nanog (clone: D2A3) antibodies for human iPSC and mouse ESC.

FIG. 6C depicts an embodiment of immunostaining of mouse-specific Oct4 protein in human iPSC (GFP-positive) co-culture with mouse ESC (tdTomato-positive) or in human iPSC only. A representative region at higher magnification outlined by dashed rectangles is also shown in insets. Arrows indicate the human iPSC weakly positive for mouse Oct4 proteins.

FIG. 6D depicts an embodiment of immunostaining of mouse-specific Nanog protein in human iPSC (GFP-positive) co-culture with mouse ESC (tdTomato-positive) or in human iPSC only. A representative region at higher magnification outlined by dashed rectangles is also shown in insets. Arrows indicate the human iPSC weakly positive for mouse Nanog proteins.

### DETAILED DESCRIPTION

Described herein are cell compositions comprising an acceptor cell and a donor cell, where the acceptor cell and donor cell are pluripotent stem cells. In some embodiments, the acceptor cell is a primed pluripotent stem cell and the donor cell is a naïve pluripotent stem cell. In some embodiments, direct cell-to-cell contact between the acceptor cell and donor cell reprograms the acceptor cell from a primed state to a naïve state. In some embodiments, but without being limited by any mechanism of action, this reprogramming may occur through tunneling nanotubes. In some embodiments, the acceptor cell and donor cell are the same species (allogeneic), such as human. In other embodiments, the acceptor cell and donor cell are different species (xenogeneic). For example, the acceptor cell is a human cell and the donor cell is a mouse cell. Also described herein are methods of preparing reprogrammed naïve acceptor cells by contacting said acceptor cells to naïve donor cells.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and means a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at leak about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning in light of the specification and refers to the performance of a method inside living organisms, usually animals, mammals, including humans, and plants, as opposed to a tissue extract or dead organism.

As used herein, “ex vivo” is given its plain and ordinary meaning in light of the specification and refers to the performance of a method outside a living organism with little alteration of natural conditions.

As used herein, “in vitro” is given its plain and ordinary meaning in light of the specification and refers to the performance of a method outside of biological conditions, e.g., in a petri dish or test tube.

The terms “nucleic acid” or “nucleic acid molecule” as used herein have their plain and ordinary meaning as understood in light of the specification and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

RNA is a nucleic acid polymer molecule that plays a wide range of functions in biological systems. Messenger RNA is responsible for the expression of proteins derived from the sequence information stored in genomic DNA. During transcription, a pre-mRNA transcript is processed (e.g. intron splicing, 5′ capping, polyadenylation) and exported from the nucleus as a mature mRNA, which is free-floating through the cytoplasm until bound to a ribosome for translation. Other RNA molecules, including but not limited to noncoding RNA (ncRNA), antisense RNA (asRNA), long noncoding RNA (lncRNA), microRNA (miRNA), Piwi-interacting RNA (piRNA), small interfering RNA (siRNA), or short hairpin RNA (shRNA), or combinations thereof, play large roles in gene regulation, typically through binding and subsequent degradation or inactivation of complementary mRNA and pathways such as the RNA-induced silencing complex (RISC). This knowledge had led to a rich toolset by which to engineer cells to transiently express (mRNA) or downregulate expression of proteins by transfecting synthesized or isolated RNA molecules. Similarly, transport of RNA molecules during inter-cellular transfer (e.g. through TNTs or microvesicles) imparts considerable effects in recipient cells.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein refers to a sequence being after the 3′-end of a previous sequence, on the strand containing the encoding sequence (sense strand) if the nucleic acid is double stranded. The term “upstream” on a nucleic acid as used herein refers to a sequence being before the 5′-end of a subsequent sequence, on the strand containing the encoding sequence (sense strand) if the nucleic acid is double stranded. The term “grouped” on a nucleic acid as used herein refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein refers to a sequence being after the C-terminus of a previous sequence. The term “upstream” on a polypeptide as used herein refers to a sequence being before the N-terminus of a subsequent sequence.

The term “purity” of any given substance, compound, or material as used herein has its plain and ordinary meaning as understood in light of the specification and refers to the actual abundance of the substance, compound, or material relative to the expected abundance. For example, the substance, compound, or material is, is about, is at least, is at least about, is not more than, or is not more than about, 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

### Stem Cells

The term “totipotent stem cells” (also known as omnipotent stem cells) as used herein has its plain and ordinary meaning as understood in light of the specification and refer to stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs),” also commonly abbreviated as ES cells, as used herein has its plain and ordinary meaning as understood in light of the specification and refers to cells that are pluripotent and derived from the inner cell mass of the blastocyst, an early-stage embryo. For purpose of the present invention, the term “ESCs” is used broadly sometimes to encompass the embryonic germ cells as well.

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (POU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with OCT4, SOX2, NANOG, and LIN28. Genes whose expression are induced in iPSCs include but are not limited to Oct-3/4 (POU5F1); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc Nanog, LIN28, Tert, Fbx15, ERas, ECAT15-1, ECAT15-2, Tcl1, β-Catenin, ECAT1, Esg1, Dnmt3L, ECAT8, Gdf3, Fth117, Sal14, Rex1, UTF1, Stella, Stat3, Grb2, Prdm14, Nr5a1, Nr5a2, or E-cadherin, or any combination thereof.

The term “precursor cell” as used herein has its plain and ordinary meaning as understood in light of the specification and encompasses any cells that can he used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC).

In some embodiments, one step is to obtain stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. Human embryonic stem cells H9 (H9-hESCs) are used in the exemplary embodiments described in the present application, but it would be understood by one of skill in the art that the methods and systems described herein are applicable to any stem cells.

Additional stem cells that can be used in embodiments in accordance with the present invention include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Exemplary embryonic stem cells that can be used in embodiments in accordance with the present invention include but are not limited to SA01 (SA001); SA02 (SA002); ES01 (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UCO1 (HSF1); UC06 (HSF6); WA01 (HI); WA07 (H7); WA09 (H9); WA13 (H13); WA14 (H14). Exemplary human pluripotent cell lines include but are not limited to TkDA3-4, 1231A3, 317-D6, 317-A4, CDH1, 5-T-3, 3-34-1, NAFLD27, NATLD77, NAFLD150WD90, WD91, WD92, L20012, C213, 1383D6, FE, or 317-12 cells.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

In some embodiments, an adenovirus can be used to transport the requisite pluripotent factors into a cell, resulting in iPSCs substantially identical to embryonic stem cells. Since the adenovirus does not combine any of its own genes with the targeted host, the danger of creating tumors is eliminated. In some embodiments, non-viral based technologies are employed to generate iPSCs. In some embodiments, reprogramming can be accomplished via plasmid without any virus transfection system at all, although at very low efficiencies. In other embodiments, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some embodiments, the expression of pluripotency induction genes can also be increased by treating somatic cells with FGF2 under low oxygen conditions.

The term “naïve” as used herein has its plain and ordinary meaning as understood in light of the specification and refers to a state of pluripotent stem cells during the early steps of development, such as those that comprise the inner cell mass or epiblast during embryogenesis (e.g. around days 4-9 following fertilization in humans). These cells can be easily cultured or genetically engineered, proliferate rapidly, have high single cell clonogenicity, and can be induced to differentiate efficiently and to tissues of all three germ layers; consequently, these cells can be used to form chimeras. In culture, these cells typically grow as domed colonies and rely on LIE for maintenance and growth. They exhibit global hypomethylation of DNA and have not undergone X chromosome inactivation. A classic example of naïve stem cells are mouse ESCs, such as those from the inner cell mass of preimplantation embryos, and mouse iPSCs. Naïve transcription factors or other associated proteins include but are not limited to KLF4, KLF5, KLF17, TFCP2L1, DNMT3L, DPPA3, DPPA5, PRDM14, SALL4, ESRRB, TFAP2C, or TBS3, or any combination thereof. Naïve cell surface markers include but are not limited to CD7, CD75, CD77, CD130, or F11R, or any combination thereof. In some embodiments, a “naïve” cell is one that expresses at least one, or at least three, or each of the following transcription factors or other associated proteins: KLF4, KLF5, KLF17, TFCP2L1, DNMT3L, DPPA3, DPPA5, PRDM14, SALL4, ESRRB, TFAP2C, and TBS3, or has greater expression of at least one, or at least three, or each of KLF4, KLF5, KLF17, TFCP2L1, DNMT3L, DPPA3, DPPA5, PRDM14, SALL4, ESRRB, TFAP2C, and TBS3 relative to a “primed” cell. In some embodiments, a “naïve” cell is one that expresses at least one, or at least three, or each of the following transcription factors or associated proteins: DPPA, TFCP2L1, DNMT3L, KLF4 or KLF17, or has greater expression of at least one, or at least three, or each of the proteins DPPA, TFCP2L1, DNMT3L, KLF4, and KLF17 relative to a “primed” cell. In some embodiments a “naïve” cell is one that expresses at least one, or at least three, or each of the following cell surface markers: CD7, CD75, CD77, CD130, and F11R, or has greater expression of at least one, or at least three, or each of the proteins CD7, CD75, CD77, CD130, and F11R relative to a “primed” cell. In some embodiments, a “naïve” cell is one that expresses at least one or each of the following cell surface markers: CD130 and CD77, or has greater expression of at least one or each of the proteins CD130 and CD77 relative to a “primed” cell. In some embodiments a “naïve” cell is one that expresses at least one, or at least three, or each of the following transcription factors, cell surface markers or other associated proteins: KLF4, KLF5, KLF17, TFCP2L1, DNMT3L, DPPA3, DPPA5, PRDM14, SALL4, ESRRB, TFAP2C, TBS3, CD7, CD75, CD77, CD130, and F11R, or has greater expression of at least one, or at least three, or each of the proteins: KLF4, KLF5, KLF17, TFCP2L1, DNMT3L, DPPA3, DPPA5, PRDM14, SALL4, ESRRB, TFAP2C, TBS3, CD7, CD75, CD77, CD130, and F11R relative to a “primed” cell. In some embodiments, a “naïve” cell is one that expresses at least one, or at least three, or each of the following transcription factors, cell surface markers, or other associated proteins: DPPA3, CP2L1, DNMT3L, KLF4, KLF17, CD130, and CD77, or has greater expression of at least one, or at least three, or each of the proteins: DPPA3, TFCP2L1, DNMT3L, KLF4, KLF17, CD130 and CD77 relative to a “primed” cell. In some embodiments, the “naïve” cell does not express one or more (e.g. at least 1, 3, 5) of the following “primed” transcription factors or other associated proteins: ZIC2, ZIC3, OTX2, DUSP6, THY1, FOXA2, or XIST, or has reduced expression of one or more (e.g. at least 1, 3, 5) of ZIC2, ZIC3, OTX2, DUSP6, THY1, FOXA2, or XIST relative to a “primed” cell. In some embodiments, the “naïve” cell does not express one or more (e.g. at least 1) of DUSP6 or THY1 or has reduced expression of one or more (e.g. at least 1) of DUSP6 or THY1 relative to a “primed” cell. In some embodiments, the “naïve” cell does not express one or more (e.g. at least 1, 3, 5) of the following “primed” cell surface markers: CD24, CD57, CD90, SSEA4, or HLAABC, or has reduced expression of one or more (e.g. at least 1, 3, 5) of CD24, CD57, CD90, SSEA4, or HLAABC relative to a “primed” cell. In some embodiments, the “naïve” cell does not express one or more (e.g. at least 1) of CD90 or HLAABC, or has reduced expression of one or more (e.g. at least 1) of CD90 or HLAABC relative to a “primed” cell. In some embodiments, the “naïve” cell does not express one or more (e.g. at least 1, 3, 5, 10) of the following proteins: ZIC2, ZIC3, OTX2, DUSP6, THY1, FOXA2, XIST, CD24, CD57, CD90, SSEA4, or HLAABC, or has reduced expression of one or more (e.g. at least 1, 3, 5, 10) of the following proteins: ZIC2, ZIC3, OTX2, DUSP6, THY1, FOXA2, XIST, CD24, CD57, CD90, SSEA4, or HLAABC compared to a “primed” cell. In some embodiments, the “naïve” cell refers to a “primed” cell that has been reprogrammed to a naïve state according to one of the methods described herein, and the relative expression of one or more (e.g. at least 1, 3, 5, 10) of the proteins listed herein for the “naïve” cell is in comparison to when it was a “primed” cell (i.e. in a “primed” state) before the step of reprogramming according to one of the methods described herein.

The term “primed” as used herein has its plain and ordinary meaning as understood in light of the specification and refers to a state of pluripotent stem cells that represent later steps of development, such as those that comprise the three primary germ layers (ectoderm, mesoderm, endoderm) following differentiation of the epiblast during embryogenesis (e.g. after day 9 after fertilization in humans). These cells are more difficult to genetically engineer, have low single cell clonogenicity, and tend to differentiate into cell lineages belonging to any one specific germ layer among the three germ layers, thereby making them unamenable for the formation of chimeras. In culture, these cells typically grow as flat monolayers and rely on Activin and or FGF2 for maintenance and growth. They exhibit global DNA hypermethylation of DNA and have undergone or are in the process of undergoing X chromosome inactivation. Primed stem cells typically include isolated human ESCs, human iPSCs, and mouse epiblast stem cells (EpiSCs). Primed transcription factors or other associated proteins include but are not limited to ZIC2, ZIC3, OTX2, DUSP6, THY1, FOXA2, or XIST, or any combination thereof. Primed cell surface markers include but are not limited to CD24, CD57, CD90, SSEA4, or HLAABC, or any combination thereof. In some embodiments, a “primed” cell is one that expresses at least one, or at least three, or each of the following “primed” transcription factors or other associated proteins: ZIC2, ZIC3, OTX2, DUSP6, THY1, FOXA2, or XIST, or has greater expression of one or more of ZIC2, ZIC3, OTX2, DUSP6, THY1, FOXA2, or XIST relative to a “naïve” cell (e.g. at least 1, 3, 5). In some embodiments, the “primed” cell is one that expresses one or more of DUSP6 or THY1 or has greater expression of one or more of DUSP6 or THY1 relative to a “naïve” cell (e.g. at least 1). In some embodiments, the “primed” cell expresses one or more of the following “primed” cell surface markers: CD24, CD57, CD90, SSEA4, or HLAABC, or has greater expression of one or more of CD24, CD57, CD90, SSEA4, or HLAABC relative to a “naïve” cell (e.g. at least 1, 3, 5). In some embodiments, the “primed” cell expresses one or more of CD90 or HLAABC, or has greater expression of one or more of CD90 or HLAABC relative to a “naïve” cell (e.g. at least 1). In some embodiments, the “primed” cell expresses one or more of the following proteins: ZIC2, ZIC3, OTX2, DUSP6, THY1, FOXA2, XIST, CD24, CD57, CD90, SSEA4, or HLAABC, or has greater expression of one or more of the following proteins: ZIC2, ZIC3, OTX2, DUSP6, FOXA2, XIST, CD24, CD57, CD90, SSEA4, or HLAABC compared to a “naïve” cell (e.g. at least 1, 3, 5). In some embodiments, a “primed” cell is one that does not express one or more of the following “naïve” transcription factors or other associated proteins: KLF4, KLF5, KLF17, TFCP2L1, DNMT3L, DPPA3, DPPA5, PRDM14, SALL4, ESRRB, TFAP2C, and TBS3, or has reduced expression of one or more of the proteins: KLF4, KLF5, KLF17, TFCP2L1, DNMT3L, DPPA3, DPPA5, PRDM14, SALL4, ESRRB, TFAP2C, and TBS3 relative to a “naïve” cell (e.g. at least 1, 3, 5, 10). In some embodiments, a “primed” cell is one that does not express one or more of the following “naïve” transcription factors or associated proteins: DPPA, TFCP2L1, DNMT3L, KLF4, or KLF17, or has reduced expression of one or more of the following proteins: DPPA, TFCP2L1, DNMT3L, KLF4, or KLF17 relative to a “naïve” cell (e.g. at least 1, 3, 5). In some embodiments, a “primed” cell is one that does not express one or more of the following cell surface markers: CD7, CD75, CD77, CD130, or F11R, or has reduced expression of one or more of the following proteins: CD7, CD75, CD77, CD130, or F11R relative to a “naïve” cell (e.g. at least 1, 3, 5). In some embodiments, a “primed” cell is one that does not express one or more of the proteins CD130 or CD77, or has a reduced expression of one or more of the proteins CD130 or CD77 relative to a “naïve” cell (e.g. at least 1). In some embodiments, a “primed” cell is one that does not express one or more of the following transcription factors, cell surface markers, or other associated proteins: KLF4, KLF5, KLF17, TFCP2L1, DNMT3L, DPPA3, DPPA5, PRDM14, SALL4, ESRRB, TFAP2C, TBS3, CD7, CD75, CD77, CD130, or F11R, or has reduced expression of one or more of the proteins: KLF4, KLF5, KLF17, TFCP2L1, DNMT3L, DPPA3, DPPA5, PRDM14, SALL4, ESRRB, TFAP2C, TBS3, CD7, CD75, CD77, CD130, or F11R relative to a “naïve” cell (e.g. at least 1, 3, 5, 10). In some embodiments, a “primed” cell is one that does not express one or more of the following transcription factors, cell surface markers, or other associated proteins: DPPA3, TFCP2L1, DNMT3L, KLF4, KLF17, CD130, or CD77, or has a reduced expression of one or more of the proteins: DPPA3, TFCP2L1, DNMT3L, KFL4, KLF17, CD130 or CD77 relative to a “naïve” cell (e.g. at least 1, 3, 5). In some embodiments, the “primed” cell refers to a cell that has not yet been reprogrammed to a naïve state according to one of the methods described herein, and the relative expression of one or more (e.g. at least 1, 3, 5, 10) of the proteins listed herein for the “primed” cell is in comparison to the expression level seen in the cell after the step of reprogramming to a “naïve” state according to one of the methods described herein.

To utilize pluripotent stem cells effectively for purposes such as stem cell research, disease modeling, drug screening, and cell-based therapies, using naïve stem cells has shown to be more preferable, and therefore there has been significant effort to transform primed stem cells to a naïve state. Previously developed techniques to shift iPSCs to a naïve state generally involve genetic manipulation to express naïve factors (e.g. KLF4, KLF2) or the use of small molecule compounds, including but not limited to glycogen synthase kinase 3β (GSK3) inhibitors (e.g. CHIR99021), mitogen-activated protein kinase ([MAPK], also known as extracellular signal-regulated kinases [ERK1/2]) inhibitors (e.g. PD0325901), leukemia inhibitor factor (LIF), c-Jun N-terminal kinase (JNK) inhibitors (e.g. SP600125), p38 inhibitors (e.g. SB203580), Rho kinase (ROCK) inhibitors (e.g. Y-27632), protein kinase C (PKC) inhibitors (e.g. Gö 6983), bone morphogenic protein (BMP) inhibitors (e.g. dorsomorphin), basic fibroblast growth factor (bFGF, FGF-2), Activin A, ascorbic acid, cAMP activator (e.g. forskolin), TGF-β, or TGF-β inhibitors (e.g. A83-01), or any combination thereof. In some embodiments, the cells are grown in 2i medium, which may comprise a GSK3 inhibitor and a MAPK inhibitor. In some embodiments, the cells are grown in mTeSR, RSet, naïve human stem cell (NHSM), T2iLGö, tt2iLGö, 5i, 4i, 3i, or feeder-independent naïve embryonic (FINE) medium, which may comprise LIF, MAPK. inhibitor, GSK3 inhibitor, JNK inhibitor, p38 inhibitor, bFGF or TGF-β, or any combination thereof. In some embodiments, the stem cells are not grown with a feeder cell substrate. In some embodiments, the stem cells are grown with a feeder cell substrate. Additional information about transforming stem cells to a naïve state can be found in Collier et al (2018) and Kumari (2016), each of which are hereby expressly incorporated by reference in its entirety.

The term “naïve maintenance medium” as used herein has its plain and ordinary meaning as understood in light of the specification and refers to a growth medium that supports naïve pluripotent stem cells and keeps the naïve pluripotent stem cells in a naïve state. The naïve maintenance medium may be any medium disclosed herein, such as PXGL medium, N2B27 medium, N2 medium, tt2iLGö medium, 2i medium, or 2i medium with gelatin. The naïve maintenance medium may also be supplemented with any of the small molecule compounds, inhibitors, activators, or growth factors described herein, for example, GSK3 inhibitors, MAPK inhibitors, LIF, JNK inhibitors, p38 inhibitors, ROCK inhibitors, PKC inhibitors, BMP inhibitors, bFGF, Activin A, ascorbic acid, cAMP activators, TGF-β or TGF-β inhibitors. In some embodiments, the naïve maintenance medium allows the naïve pluripotent stem cells to survive or proliferate and keep their naïve state for a period of culture that is, is about, is at least, is at least about, is not more than, or is not more than about, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days of culture. In some embodiments, the percentage of naïve pluripotent stem cells that maintain their naïve state after a certain period of culture is, is about, is at least, is at least about, is not more than, or is not more than about, 40%, 45%, 50%, 55%, 60%, 65%, 70%, 75%, 80%, 85%, 90%, 95% or 99%, or any percentage within a range defined by any two of the aforementioned percentages, for example, 40% to 99%, 50% to 90%, 60% to 80%, 40% to 70% or 60% to 99%. In some embodiments, the naïve maintenance medium can be used to support primed pluripotent stem cells as well. In some embodiments, the naïve maintenance medium reprograms primed pluripotent stem cells into a naïve state after a period of culture that is, is about, is at least, is at least about, is not more than, or is not more than about, for example, after 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days of culture. In other embodiments, the naïve maintenance medium does not support primed pluripotent stem cells, where the number, viability, or clonogenicity of the primed pluripotent stem cells decreases over time. In some embodiments, the percentage of primed pluripotent stem cells that lose viability or clonogenicity after a certain period of culture is, is about, is at least, is at least about, is not more than, or is not more than about, 10%, 15%, 20%, 25%, 30%, 35%, 40%, 45%, 50%, 55%, 60%, 65%, 70%, 75%, 80%, 85%, 90%, 95% or 99%, or any percentage within a range defined by any two of the aforementioned percentages, for example, 10% to 99%, 30% to 80%, 40% to 70%, 10% to 60% or 40% to 99%.

The term “chemical reset (cR) cell” as used herein has its plain and ordinary meaning as understood in light of the specification and refers to primed pluripotent stem cells that are reprogrammed to a naïve state with the use of historic deacetylase (HDAC) inhibitors. Exemplary HDAC inhibitors used for chemical reset include but are not limited to valproic acid, sodium butyrate, vorinostat, panobinostat, belinostat, givinostat, dacinostat, PCI-24781, CHR-3996, JNJ-26481585, SB939, AR-42, ACY-1215, romidepsin, alpha-ketomide, HKI 46F08, phenylbutyrate, pivanex, entinostat, mocetinostat, tacedinaline, or CUDC-101, or any combination thereof. Additional information about cR cells can be found in Guo, G et al. (2017). Epigenetic resetting of human pluripotency. Development. 144, 2748-2763, hereby expressly incorporated by reference in its entirety.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), mitomycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are used during the proliferation of the target stem cells. In some embodiments, feeder cells are not used during the proliferation of the target stem cells.

The term “inter-cellular transfer” as used herein has its plain and ordinary meaning as understood in light of the specification and refers to the transportation of biological material from one cell to another. While the plasma membrane typically acts as a barrier, mechanisms exist for cells to exchange material including but not limited to fluids, salts, nutrients, sugars, small molecule compounds, organelles, mitochondria, endosomes, vesicles, proteins, polypeptides, peptides, nucleic acids, DNA, or RNA, or any combination thereof. RNA may include mRNA, miRNA, siRNA, shRNA, or other types of RNA disclosed herein or known in the art and may result in expression of exogenous proteins or downregulation of gene expression through endogenous silencing pathways. Without being limited by any mechanism of action, inter-cellular transfer may occur through tunneling nanotubes (TNTs) or cytonemes, which are long actin-containing membrane protrusions that connect two or more cells and facilitate transport. As disclosed herein, these TNTs may be selective for a certain category of biological material (e.g. by size, polarity, charge, stability, etc) and even for certain species of one category (e.g. one RNA is transferred more efficiently than another). Pro-inflammatory stimuli, such as lipopolysaccharide (LPS) or IFN-γ, reduces TNT formation. Another method of inter-cellular transfer is through microvesicles and exosomes. These small membrane-bound vesicles have been shown to be able to carry RNA such as mRNA and miRNA. As shown herein, these two types of inter-cellular transfer can be differentiated by testing conditioned media or through a transwell assay, which will permit transfer of free floating microvesicles and exosomes, but not allow direct cell-to-cell contact, which is necessary for TNTs.

Some embodiments described herein relate to pharmaceutical compositions that comprise, consist essentially of, or consist of an effective amount of a cell composition described herein and a pharmaceutically acceptable carrier, excipient, or combination thereof. A pharmaceutical composition described herein is suitable for human and/or veterinary applications.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and is intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” have their plain and ordinary meaning as understood in light of the specification and can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more (e.g. at least 1, 3, 5, 10) of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The invention is generally disclosed herein using affirmative language to describe the numerous embodiments. The invention also includes embodiments in which subject matter is excluded, in full or in part, such as substances or materials, method steps and conditions, protocols, or procedures.

### Methods of Making and Compositions of Reprogrammed Naïve Stem Cells

Described herein are compositions comprising reprogrammed naïve stem cells and methods of preparing the same. These reprogrammed naïve stem cells exhibit characteristics seen in the naïve stein cells that are involved during embryogenesis (i.e. the cells that make up the inner cell mass and epiblast of the developing embryo before differentiation of the epiblast into the three germ layers) or the naïve stem cells that are reprogrammed from primed stem cells through other methods known in the art, such as transgenic expression of naïve transcription factors or the use of small molecule compounds, inhibitors, or activators that transition the primed stem cell to a naïve state. These characteristics include, but are not limited to, expression of naïve transcription factors, expression of naïve cell surface markers, dome-like cell colony morphology, ability to persist and grown in cell culture media known to be incompatible with primed stem cells, and modification in chromatin state to allow access to genes encoding transcription factors, other proteins and non-coding RNA associated with the naïve state while simultaneously downregulating expression of transcription factors, other proteins, and non-coding RNA associated with a primed or somatic state. These reprogrammed naïve stem cells are not necessarily totipotent stem cells, as they do not have the capability to form extraembryonic cells and tissues.

Described herein are methods of reprogramming a cell from a primed state to a naïve state. The methods comprise contacting in vitro an acceptor cell with a donor cell. In some embodiments, the contacting causes transfer of an intra-cellular component from the donor cell to the acceptor cell. As shown herein in some embodiments, this reprogramming phenomenon is induced through direct cell-to-cell contact between one cell (the “donor cell”) and a second cell (the “acceptor cell”). Without being limited by any mechanism of action, it is believed that the donor cell and acceptor cell form cytoplasmic bridges through long protrusions called tunneling nanotubes (TNTs) or cytonemes. These TNTs permit the transfer of cellular material, including RNA (including mRNA, ncRNA, lncRNA, miRNA, piRNA, siRNA, or shRNA). It is shown that among this mRNA, at least 491 donor-derived naïve state-related transcription factors are found to be transferred to the acceptor cell. The presence of these mRNA that encode for naïve transcription factors in the acceptor cell is potent enough to induce reprogramming from a primed state to a naïve state. The use of inflammatory stimuli molecules, such as LPS, prevents the formation of TNTs and consequently inhibits this naïve reprogramming. Donor cell-conditioned medium or a transwell separating the two cell populations also fails to induce naïve reprogramming in the acceptor cells, demonstrating that other known inter-cellular transfer mechanisms (e.g. microvesicles, exosomes) are not sufficient for reprogramming and that direct cell-to-cell contact and formation of TNTs is necessary. In some embodiments, this process of naïve reprogramming is not done with viruses. In some embodiments, this process of naïve reprogramming is not done with adeno-associated viruses or lentiviruses. In some embodiments, this process of naïve reprogramming is not done with transfection, transduction, or electroporation.

The acceptor cell herein is a cell that receives genetic and other cellular material from a donor cell by co-culture with the donor cell wherein the donor and acceptor cells are in direct contact, presumably but without being limited by any mechanism of action, through TNTs. In some embodiments, the acceptor cell is a pluripotent stem cell. In some embodiments, the acceptor cell is an iPSC. In some embodiments, the acceptor cell is a primed pluripotent stem cell. In some embodiments, the acceptor cell is a primed iPSC. In some embodiments, the acceptor cell is a mammalian cell. In some embodiments, the acceptor cell is a mouse cell. In some embodiments, the acceptor cell is a human cell. In some embodiments, the acceptor cell is a human PSC. In some embodiments, the acceptor cell is an hiPSC. In some embodiments, the acceptor cell is a primed hiPSC. In some embodiments, the acceptor cell is a cell in a primed state. In some embodiments, the acceptor cell expresses primed transcription factors and/or primed cell surface markers. In some embodiments, the acceptor cell is of the TkDA3-4, 1231A3, 317-D6, 317-A4, CDH1, 5-T-3, 3-34-1, NAFLD27, NAFLD77, NAFLD150, WD90, WD91, WD92, L20012, C213, 1383D6, FF, or 317-12 cell line. After directly contacting the acceptor cell with a donor cell, the acceptor cell receives material such as mRNA, ncRNA, proteins, and organelles from the donor cell. In some embodiments, the contacting occurs in a cell culture. In some embodiments, the contacting takes place over a number of days that is, is about, is at least, is at least about, is not more than, is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days, or a range.

In some embodiments, the number of genes that are expressed in the acceptor cell after contacting with the donor cell that were not expressed prior to contacting is, is about, is at least, is at least about, is not more than, or is not more than about, 100, 200, 300, 400, 500, 1000, 2000, 3000, 4000, 5000, 6000, 6382, 7000, 8000, 9000 additional genes, or a range defined by any two of the preceding values, for example 100-9000, 2000-8000, 4000-8000, 100-500, or 300-2000. In some embodiments the additional genes that are expressed in the acceptor cell are expressed from exogenous mRNA received from the donor cell. In some embodiments the exogenous mRNA from the donor cell encodes for naïve transcription factors. In some embodiments the exogenous mRNA from the donor cell encodes for naïve cell surface markers. In some embodiments, the additional genes that are expressed include genes that are involved in protein targeting to membrane, symbiont process, translational initiation, and RNA processing and localization. In some embodiments, the acceptor cell and donor cell are of different species, and the exogenous mRNA is xenogeneic. In some embodiments, the acceptor cell and donor cell are of the same species, and the exogenous triRNA is allogeneic or autologous. In some embodiments, the acceptor cell and donor cell have different genomic DNA, and the exogenous mRNA differs in genetic sequence to the genomic DNA of the acceptor cell. In some embodiments, some mRNA, ncRNA, or proteins are transferred from the donor cell to the acceptor cell more favorably than other mRNA, ncRNA, or proteins. For example, Wnt1, Wnt5a, Hoxa11, Ffgl3 are highly expressed genes in the donor cell (i.e. multiple copies of the mRNA are present) but do not get transferred to the acceptor cell with high efficiency.

In some embodiments, after contact with the donor cell, the acceptor cell transitions from a primed state to a naïve state. In some embodiments, after contact with the donor cell, the acceptor cell expresses or upregulates expression of one or more naïve pluripotency markers or transcription factors. In some embodiments, after contact with the donor cell, the acceptor cell expresses or upregulates expression of one or more (e.g. at least 1, 3, 5 or 10) naïve pluripotency markers or transcription factors KLF4, KLF5, KLF17, TFCP2L1, DNMT3L, DPPA3, DPPA5, PRDM14, SALL4, ESRRB, TFAP2C, or TBS3. In some embodiments, after contact with the donor cell, the acceptor cell expresses or upregulates expression of one or more (e.g. at least 1, 3 or 5) naïve pluripotency markers or transcription factors DPPA3, DFCP2L1, DNMT3L, KLF4, or KLF17. In some embodiments, after contact with the donor cell, the acceptor cell expresses or upregulates expression of one or more naïve cell surface markers. In some embodiments, after contact with the donor cell, the acceptor cell expresses or upregulates expression of one or more, or all of the naïve cell surface markers CD130, CD77, CD7, CD75, or F11R (e.g. at least 1, 3, 5). In some embodiments, after contact with the donor cell, the acceptor cell expresses or upregulates expression of one or more (e.g. at least 1) naïve cell surface markers CD130 or CD77. In some embodiments, after contact with the donor cell, the acceptor cell expresses or upregulates expression of one or more (e.g. at least 1, 3, 5, 10) naïve pluripotency markers, transcription factors, or cell surface markers KLF4, KLF5, KLF17, TFCP2L1, DNMT3L, DPPA3, DPPA5, PRDM14, SALL4, ESRRB, TFAP2C, TBS3, CD130, CD77, CD7, CD75, or F11R. In some embodiments, after contact with the donor cell, the acceptor cell expresses exogenous genes from the donor cell. In some embodiments, after contact with the donor cell, the acceptor cell expresses exogenous actin from the donor cell. In some embodiments, after contact with the donor cell, the acceptor cell expresses exogenous NANOG from the donor cell.

In some embodiments, after contact with the donor cell, the acceptor cell does not express or downregulates expression of one or more primed pluripotency markers or transcription factors. In some embodiments, after contact with the donor cell, the acceptor cell does not express or downregulates expression of one or more (e.g. at least 1, 3, 5) primed pluripotency markers or transcription factors ZIC2, ZIC3, OTX2, DUSP6, THY1, FOXA2, or XIST. In some embodiments, after contact with the donor cell, the acceptor cell does not express or downregulates expression of the primed pluripotency marker or transcription factor DUSP6 or THY1. In some embodiments, after contact with the donor cell, the acceptor cell does not express or downregulates expression of one or more primed cell surface markers. In some embodiments, after contact with the donor cell, the acceptor cell does not express or downregulates expression of one or more (e.g. at least 1, 3, 5) primed cell surface markers CD90, HLA-ABC, CD24, CD57, or SSEA4. In some embodiments, after contact with the donor cell, the acceptor cell does not express or downregulates expression of one or more (e.g. at least 1) primed cell surface markers CD90 or HLAABC. In some embodiments, after contact with the donor cell, the acceptor cell does not express or downregulates expression of one or more (e.g. at least 1, 3, 5, 10) primed pluripotency markers, transcription factors, or cell surface markers ZIC2, ZIC3, OTX2, DUSP6, THY1, FOXA2, XIST, CD90, HLA-ABC, CD24, CD57, or SSEA4.

In some embodiments, after contact with the donor cell, the acceptor cell undergoes changes in chromatin accessibility. In some embodiments, the changes in chromatin accessibility allows for expression of naïve pluripotency markers, transcription factors, or cell surface markers. In some embodiments, after contact with the donor cell, the acceptor cell undergoes changes in over half of the total open chromatin regions of its genome. In some embodiments, after contact with the donor cell, the acceptor cell undergoes changes in chromatic accessibility, wherein binding motifs for SOX2 or TFAP2C, or both, are increased in accessibility.

The donor cell herein is a cell that provides genetic and other cellular material to an acceptor cell by direct cell to cell contact. While not being limited to any particular mechanism, it is believed that the transfer is through TNTs. In some embodiments, the donor cell is a pluripotent stem cell. In some embodiments, the donor cell is an iPSC. In some embodiments, the donor cell is a naïve PSC. In some embodiments, the donor cell is a mammalian cell. In some embodiments, the donor cell is a mouse cell. In some embodiments, the donor cell is a human cell. In some embodiments, the donor cell is a human PSC. In some embodiments, the donor cell is a mouse PSC. In some embodiments, the donor cell is a mouse iPSC. In some embodiments, the donor cell is a mouse ESC. In some embodiments, the donor cell is a naïve mouse ESC. After directly contacting the donor cell with acceptor cell, the donor cell provides material such as mRNA, ncRNA, proteins, and organelles to the acceptor cell. In some embodiments, the contacting occurs in a cell culture. In some embodiments, the contacting takes place over a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, or 50 days.

In some embodiments, the donor cell and acceptor cell are of different species, and the donor cell provides exogenous triRNA that is xenogeneic to the acceptor cell. In some embodiments, the donor cell and acceptor cell are of the same species, and the donor cell provides exogenous mRNA that is allogeneic or to the acceptor cell. In some embodiments, the donor cell and acceptor cell are from the same individual, and the donor cell provides exogenous mRNA that is autologous to the acceptor cell. In some embodiments, the donor cell and acceptor cell have different genomic DNA, and the donor cell provides exogenous mRNA to the acceptor cell that differs in genetic sequence of the acceptor cell. In some embodiments, after contacting, the acceptor cell expresses protein from the exogenous mRNA from the donor cell. In some embodiments, after contacting, the acceptor cell expresses protein from the exogenous mRNA that is xenogeneic. In some embodiments, after contacting, the acceptor cell expresses protein from the exogenous mRNA that is allogeneic. In some embodiments, the acceptor cell expresses protein from the exogenous mRNA that is autologous.

In some embodiments, after contact with the acceptor cell, the donor cell stays in a naïve state. In some embodiments, after contact with the acceptor cell, the donor cell stays in a naïve state when cultured in naïve maintenance media or naïve stem cell growth media. In some embodiments, after contact with the acceptor cell, the donor cell expresses or highly expresses one or more (e.g. at least 1, 3, 5, 10) naïve pluripotency markers, transcription factors, or cell surface markers KLF4, KLF5, KLF17, TFCP2L1, DNMT3L, DPPA3, DPPA5, PRDM14, SALL4, ESRRB, TFAP2C, TBS3, CD130, CD77, CD7, CD75, or F11R. In some embodiments, after contact with the acceptor cell, the donor cell does not express or lowly expresses one or more (e.g. at least 1, 3, 5, 10) primed pluripotency markers, transcription factors, or cell surface markers ZIC2, ZIC3, OTX2, DUSP6, THY1, FOXA2, XIST, CD90, HLA-ABC, CD24, CD57, or SSEA4. In some embodiments, after contact with the acceptor cell, the donor cell transitions from a naïve state to a primed state. In some embodiments, after contact with the acceptor cell, the donor cell does not express or downregulates expression of one or more (e.g. at least 1, 3, 5, 10) naïve pluripotency markers, transcription factors, or cell surface markers KLF4, KLF5, KLF17, TFCP2L1, DNMT3L, DPPA3, DPPA5, PRDM14, SALL4, ESRRB, TFAP2C, BS3, CD130, CD77, CD7, CD75, or F11R. In some embodiments, after contact with the acceptor cell, the donor cell expresses or upregulates expression of one or more (e.g. at least 1, 3, 5, 10) primed pluripotency markers, transcription factors, or cell surface markers ZIC2, ZIC3, OTX2, DUSP6, THY1, FOXA2, XIST, CD90, HLA-ABC, CD24, CD57, or SSEA4. In some embodiments, after contact with the acceptor cell, the donor cell comprises exogenous mRNA from the acceptor cell. In some embodiments, after contact with the acceptor cell, the donor cell comprises exogenous mRNA from the acceptor cell that is xenogeneic. In some embodiments, after contact with the acceptor cell, the donor cell comprises exogenous mRNA from the acceptor cell that is allogeneic or autologous. In some embodiments, the donor cell and acceptor cell have different genomic DNA, and the donor cell comprises exogenous mRNA from the acceptor cell that differs in genetic sequence to the genetic DNA of the donor cell. In some embodiments, the donor cell comprises exogenous actin or NANOG mRNA from the acceptor cell.

In some embodiments, the acceptor cell and donor cell are contacted or cultured at a ratio that is, is about, is at least, is at least about, is not more than, or is not more than about, 1%:99%, 5%:95%, 10%:90%, 15%:85%, 20%:80%, 25%:75%, 30%:70%, 35%:65%, 40%:60%, 45%:55%, 50%:50%, 55%:45%, 60%:40%, 65%:35%, 70%:30%, 75%:25%, 80%:20%, 85%:15%, 90%:10%, 95%:5%, or 99%:1%, or any ratio within a range defined by any two of the aforementioned ratios, for example 1%:99% to 99%:1%, 10%:90% to 90%:10%, 20%:80% to 80%:20%, 30%:70% to 70%:30%, 40%:60% to 60%:40%, 45%:55% to 55%:45%, 1%:99% to 50%:50% or 50%:50% to 99%:1%. In some embodiments, the acceptor cell and donor cell are contacted or cultured at a ratio that is, is about, is at least, is at least about, is not more than, or is not more than about, 50%:50%. In some embodiments, the acceptor cell and donor cell are contacted or cultured at a ratio that is, is about, is at least, is at least about, is not more than, or is not more than about, 20%:80%. In some embodiments, the acceptor cell and donor cell are contacted or cultured at a ratio that is, is about, is at least, is at least about, is not more than, or is not more than about, 80%:20%. In some embodiments, before contact with the acceptor cells, the donor cells are in dome-shaped colonies. In some embodiments, before contact with the donor cells, the acceptor cells are in flat monolayers. In some embodiments, after contact with the donor cells, the acceptor cells are in dome-shaped colonies. In some embodiments, the acceptor cell and donor cell are in direct contact with each other. In some embodiments, the acceptor cell and donor cell are not separated by a transwell.

In some embodiments, the acceptor cell and donor cell are contacted or cultured under hypoxic conditions. In some embodiments, the hypoxic conditions comprise, consist essentially or, or consist of a concentration of O₂ that is, is about, is at least, is at least about, is not more than, or is not more than about, 0%, 1%, 2%, 3%, 4%, 5%, 6%, 7%, 8%, 9%, 10%, 11%, 12%, 13%, 14%, 15%, 16%, 17%, 18%, 19%, or 20% O₂, or any concentration of O₂ within a range defined by any two of the aforementioned concentration, for example 0% to 20%, 3% to 10%, 4% to 6%, 0% to 5%, or 5% to 20%. In some embodiments, the hypoxic conditions comprise, consist essentially of, or consist of a concentration of O₂ that is, is about, is at leak, is at least about, is not more than, or is not more than about, 4%, 5%, or 6% O₂. In some embodiments, the hypoxic conditions comprise, consist essentially of, or consist of a concentration of O₂ that is, is about, is at least, is at least about, is not more than, or is not more than about, 5% O₂.

In some embodiments, the acceptor cell and donor cell are contacted or cultured at a temperature that is, is about, is at least, is at least about, is not more than, or is not more than about, 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., or any temperature within a range defined by any two of the aforementioned temperatures, for example, 15° C. to 50° C., 20° C. to 45° C., 25° C. to 40° C. 32° C. to 42° C. or 35° C. to 39° C. In some embodiments, the donor cell and acceptor cell are contacted or grown at a temperature that is, is about, is at least, is at least about, is not more than, or is not more than about, 37° C.

In some embodiments, before contact with the donor cell, the acceptor cell cannot persist or proliferate in naïve stem cell growth media or naïve maintenance media. In some embodiments, before contact with the acceptor cell, the donor cell can persist or proliferate in naïve stem cell growth media or naïve maintenance media. In some embodiments, after contact with the donor cell, the acceptor cell is reprogrammed and can persist or proliferate in naïve stem cell growth media or naïve maintenance media. In some embodiments, the naïve stem cell growth media or naïve maintenance media is RSet medium, naïve human stem cell (NHSM), 5i medium, 4i medium, 3i medium, feeder-independent naïve embryonic (FINE) medium, mTeSR medium, mTeSR medium with Matrigel, PXGL medium, N2B27 medium, N2 medium, T2iLGö, tt2iLGö medium, 2i medium, or 2i medium with gelatin. In some embodiments, the naïve stem cell growth media or naïve maintenance media comprises one or more (e.g. at least 1, 3, 5) of a GSK3 inhibitor, MAPK inhibitor, LIF, JNK inhibitor, p38 inhibitor, bFGF, or TGF-β. In some embodiments, the acceptor cell or donor cell or both are grown on a feeder cell substrate. In some embodiments, the acceptor cell or donor cell or both are not grown on a feeder cell substrate. In some embodiments, after contact with the donor cell, the acceptor cell is reprogrammed and can be grown without a feeder cell substrate where otherwise a feeder cell substrate would be needed. In some embodiments, the donor cell is not a chemical reset cell. In some embodiments, the acceptor cell is not a chemical reset cell. In some embodiments, the donor cell and acceptor cell are not contacted or cultured with an HDAC inhibitor. In some embodiments, the donor cell and acceptor cell are not contacted or cultured with one or more (e.g. at least 1, 3, 5) of valproic acid, sodium butyrate, vorinostat, panobinostat, belinostat, givinostat, dacinostat, PCI-24781, CHR-3996, JNJ-2648158, SB939, AR-42, ACY-1215, romidepsin, alpha-ketomide, 46F08, phenylbutyrate, pivanex, entinostat, mocetinostat, tacedinaline, or CUDC-101, or any combination thereof. In some embodiments, the donor cell is not modified by transfection, electroporation, or transduction with a virus, or any combination thereof, prior to contacting. In some embodiments, the acceptor cell is not modified by transfection, electroporation, or transduction with a virus, or any combination thereof, prior to contacting. In some embodiments, the acceptor cell is not modified to exogenously express a transcription factor prior to contacting. In some embodiments, the acceptor cell is not modified to exogenously express a naïve transcription factor prior to contacting. In some embodiments, the acceptor cell is not modified to exogenously express one or more (e.g. at least 1, 3, 5) of Oct-3/4, Sox1, Sox2, Sox3, Sox15, Klf1, Klf2, Klf4, Klf5, C-myc, L-myc, N-myc, Nanog, LIN28, Tert, Fbx15, ERas, ECAT15-1, ECAT15-2, Tel1, β-Catenin, ECAT1, Esg1, Dnmt3L, ECAT8, Gdf3, Fth117, Sal14, Rex1, UTF1, Stella, Stat3, Grb2, Prdm14, Nr5a1, Nr5a2, or E-cadherin, or any combination thereof, prior to contacting.

In some embodiments, the donor cell and acceptor cell are contacted or cultured under a stressor condition, wherein said stressor condition is selected from contact with a cell-toxic compound, hypoxia, non-physiological temperature, non-physiological pH, electroporation, or any combination thereof. In some embodiments, the donor cell or acceptor cell or both are contacted with at least one agent selected from the group consisting of resveratrol, epigallocatechin gallate (EGCG), curcumin, genistein, activin-A, Wnt-3a, sodium butylate, basic fibroblast growth factor (bFGF), oncostatin M (OSM), dexamethasone (DEX), hepatocyte growth factor (HGF), CHIR-99021, forskolin, Y-27632 (ROCK inhibitor), (s)-(−)-blebbistatin, IWP2, A83-01, LY294002, SB-431542, NVP-BHG, Cyclopamine-KAAD, PD-0325901, FGF4, LDN-193189, insulin like growth factor (IGF), hone morphogenetic protein 2 (BMP2), transforming growth factor β2 (TGF-β2), BMP4, FGF-7, platelet-derived growth factor (PDGF) β3, epidermal growth factor (EGF), exendin-4, human neuregulin (PIRG) β3, retinoic acid (RA), L-Ascorbic acid 2-phosphate (AA2P), ascorbic acid, insulin-transferrin-selenium ethanolamine solution (ITS-X), insulin, rifampicin, penicillin, streptomycin, 2-mercaptoethanol, 3-mercaptopropane-1,2-diol (thioglycerol), L-proline, L-glutamine, non-essential amino acid mixture (NEAA), sodium pyruvate, trypsin-EDTA, phosphatidylinositol (PI), interleukins, prostaglandins, and tumor necrosis factors, or any combination thereof.

In the embodiments described herein, an acceptor cell is one that receives genetic and other cellular material, and a donor cell is one that provides genetic and other cellular material. In some embodiments, the genetic and other cellular material comprises mRNA. In some embodiments, the acceptor cell receives genetic and other cellular material by being in direct contact with a donor cell. In some embodiments, the donor cell provides genetic and other cellular material by being in direct contact with an acceptor cell. In some embodiments, the acceptor cell is a primed stem cell. In some embodiments, the donor cell is a naïve stem cell. In some embodiments, the acceptor cell is a stem cell and the donor cell is a stem cell. In some embodiments, the acceptor cell is a primed stem cell and the donor cell is a naïve stem cell. In some embodiments, the acceptor cell is a primed iPSC and the donor cell is a naïve iPSC. In some embodiments, the acceptor cell is a human stem cell and the donor cell is a human stem cell. In some embodiments, the acceptor cell is a human iPSC and the donor cell is a human iPSC. In some embodiments, the acceptor cell is a primed human iPSC and the donor cell is a naïve human iPSC. In some embodiments, the acceptor cell is a human stem cell and the donor cell is a mouse stem cell. In some embodiments, the acceptor cell is a human iPSC and the donor cell is a mouse iPSC. In some embodiments, the acceptor cell is a human iPSC and the donor cell is a mouse ESC. In some embodiments, the acceptor cell is a primed hiPSC and the donor cell is a naïve mouse iPSC. In some embodiments, the acceptor cell is a human iPSC and the donor cell is a naïve mouse ESC. In some embodiments, the acceptor cell is a naïve human iPSC and the donor cell is a naïve human iPSC. In some embodiments, the acceptor cell is a naïve human iPSC and the donor cell is a naïve mouse iPSC. In some embodiments, the acceptor cell is a naïve human iPSC and the donor cell is a naïve mouse ESC.

Reprogrammed naïve stem cells, such as human reprogrammed naïve stem cells, can be prepared by the methods provided herein, including in the Examples (i.e. co-culturing in direct contact with a naïve stem cell population). Reprogrammed naïve stem cells prepared according to these methods exhibit superior properties compared to alternative primed-to-naïve reprogramming protocols. These alternative protocols generally involve transgenic expression of pluripotency transcription factors or the use of small molecule compounds or growth factors such as leukemia inhibitory factor (LIF), basic fibroblast growth factor (bFGF, FGF-2), transforming growth factor β (TGF-β), c-Jun N-terminal kinase (JNK), Rho kinase (ROCK), bone morphogenetic protein (BMP), Activin A, or combinations thereof or inhibitors or activators or combinations of inhibitors or activators thereof. These alternative protocols may also require the use of feeder cells, such as mouse fibroblasts, mouse embryonic fibroblasts, mouse STO cells, mouse SNL 76/7 cells, human fibroblasts, human foreskin fibroblasts, human dermal fibroblasts, human amniotic mesenchymal cells, or human umbilical cord mesenchymal cells. In some embodiments of the present methods and compositions, the culture does not contain feeder cells in addition to the acceptor and donor cells.

The methods provided herein to prepare reprogrammed naïve stem cells may be faster than the previous protocols. For example, naïve transcription factors or markers can be observed in the reprogrammed acceptor cells a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days after contacting with the donor cells. The methods provided herein may be done without the need for one or more small molecule compounds or growth factors, or without the use of feeder cells.

The reprogrammed naïve stem cells prepared according to the methods provided herein have a faster doubling time and can be differentiated into a wider range of lineages as compared to the parent primed stem cells, both in vitro and in vivo. This enables for the rapid expansion of stem cells that can eventually be used to produce desirable cell types and assemblies, such as cell cultures, tissues, and organoids. The improved differentiation ability may lead to tissue or organoids that more closely resemble animal tissue and organs.

In some embodiments, the doubling time of the reprogrammed naïve stem cells is, is about, is at least, is at least about, is not more than, or is not more than about, 5, 6, 7, 8, 9, 10, 11, 12, 13, 14, 15, 16, 17, 18, 19, 20, 21, 22, 23, or 24 hours, or any time within a range defined by any two of the aforementioned times, for example 5 to 24 hours, 10 to 20 hours, 14 to 18 hours, 5 hours to 20 hours, or 15 hours to 24 hours. In some embodiments, the doubling time of the reprogrammed naïve stem cells is, is about, is at least, is at least about, is not more than, or is not more than about, 20%, 25%, 30%, 40%, 45%, 50%, 55%, 60%, 65%, 70%, 75%, 80%, 85%, 90%, 95% or 99% of the doubling time of the parent primed stem cells, or any percentage within a range defined by any two of the aforementioned doubling time percentages, for example 20% to 99%, 40% to 70%, 50% to 60%, 20% to 60%, or 40% to 99%.

In some embodiments, the single cell clonogenicity of the reprogrammed naïve stem cells is, is about, is at leak, is at least about, is not more than, or is not more than about, 100%, 110%, 120%, 130%, 140%, 150%, 160%, 170%, 180%, 190%, 200%, 210%, 220%, 230%, 240%, 250%, 260%, 270%, 280%, 290%, 300%, 350%, 400%, 450%, or 500% of the single cell clonogenicity of the parent primed stem cells, or any percentage within a range defined by any two of the aforementioned single cell clonogenicity percentages, for example 100% to 500%, 120% to 300%, 150% to 250%, 100% to 200%, or 150% to 500%.

In some embodiments, the reprogrammed naïve stem cells can be re-primed to a primed pluripotent stem cell state. In some embodiments, the reprogrammed naïve stem cells can be differentiated into a mesoderm cell, mesoderm lineage cell, ectoderm cell, ectoderm lineage cell, endoderm cell, or endoderm lineage cell, or any combination thereof. In some embodiments, the reprogrammed naïve stem cells can be differentiated into somatic cells, hematopoietic cells, endothelial cells, myocytes, stromal cells, bone cells, epidermal cells, epithelial cells, liver cells, hepatocytes, gastrointestinal cells, stomach cells, parietal cells, alveolar cells, pancreatic cells, neural cells, neurons, neural crest cells, melanocytes, or keratinocytes, or any combinations thereof. In some embodiments, the reprogrammed naïve stem cells can differentiate into a wider range of somatic cells compared to the parent primed stem cells.

Embodiments of the present disclosure include cell compositions comprising, consisting essentially of, or consisting of two populations of cells. In some embodiments, the cell compositions comprise, consist essentially of, or consist of a population of acceptor cells and a population of donor cells. In some embodiments, the cell compositions comprise, consist essentially of, or consist of an acceptor cell and a donor cell. In some embodiments, the acceptor cell and donor cell are induced pluripotent stem cells. In some embodiments, the acceptor cell is a human cell and the donor cell is a mouse cell. In some embodiments, the acceptor cell is a primed stem cell and the donor is a naïve stem cell. In some embodiments, the acceptor cell is transitioned from a primed state to a naïve state (reprogrammed acceptor cell or reprogrammed naïve stem cell) by being in direct contact with the donor cell. In some embodiments, the cell composition comprises, consists essentially of, or consists of a reprogrammed acceptor cell and a donor cell. In some embodiments, both the reprogrammed acceptor cell and donor cell are naïve stem cells or exhibit properties of a naïve stem cell. In some embodiments, both the reprogrammed acceptor cell and donor cell are not primed stem cells or do not exhibit properties of a naïve stem cell. Embodiments herein also include cell cultures comprising, consisting essentially of or consisting of an acceptor cell and a donor cell. In some embodiments, the cell composition or cell culture further comprises, consists essentially of, or consists of a growth medium. In some embodiments, the growth medium is a medium that supports naïve stem cells but not primed stem cells. In some embodiments, the growth medium comprises one or more small molecules, activators, or inhibitors described herein. In some embodiments, the growth medium further comprises a cryoprotectant. Embodiments herein also include methods of preparing the cell compositions or cell cultures described herein. In some embodiments, the methods comprise, consist essentially of, or consist of contacting or culturing a population of acceptor cells with a population of donor cells. In some embodiments, the methods comprise, consist essentially of, or consist of contacting or culturing an acceptor cell with a donor cell. Embodiments herein also include reprogrammed naïve stem cells or a population of reprogrammed naïve stem cells prepared according to the methods provided herein. In some embodiments, the reprogrammed naïve stem cells or population of reprogrammed naïve stem cells are distinct from reprogrammed naïve stem cells generated by other methods. In some embodiments, the reprogrammed naïve stem cells or population of reprogrammed naïve stem cells are not genetically manipulated. In some embodiments, the reprogrammed naïve stem cells or population of reprogrammed naïve stern cells are not contacted with one or more small molecules, activators, or inhibitors described herein (e.g. molecules that are used to reprogram stem cells from a primed state to a naïve state in other methods). Embodiments herein also include cell populations, cultures, tissues, organoids, or organs produced by differentiating the reprogrammed naïve stem cells or population of reprogrammed naïve stem cells. In some embodiments, the cell populations, cultures, tissues, organoids, or organs comprise, consist essentially of, or consist of reprogrammed naïve stem cells or population of reprogrammed naïve stem cells that are differentiated back to a primed state. In some embodiments, the cell populations, cultures, tissues, organoids, or organs comprise, consist essentially of, or consist of reprogrammed naïve stem cells or population of reprogrammed naïve stem cells that are differentiated to somatic cells. In some embodiments, only a percentage of reprogrammed naïve stem cells or population of reprogrammed naïve stem cells are differentiated, such as a percentage that is, is about, is at least, is at least about, is not more than, or is not more than about, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%, or any percentage of cell within a range defined by any two of the aforementioned percentages, for example 5% to 99%, 20% to 80%, 40% to 60%, 5% to 60%, or 40% to 99%.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1

### Nanotube Dependent mRNA Transfer Between Mouse and Human Cells

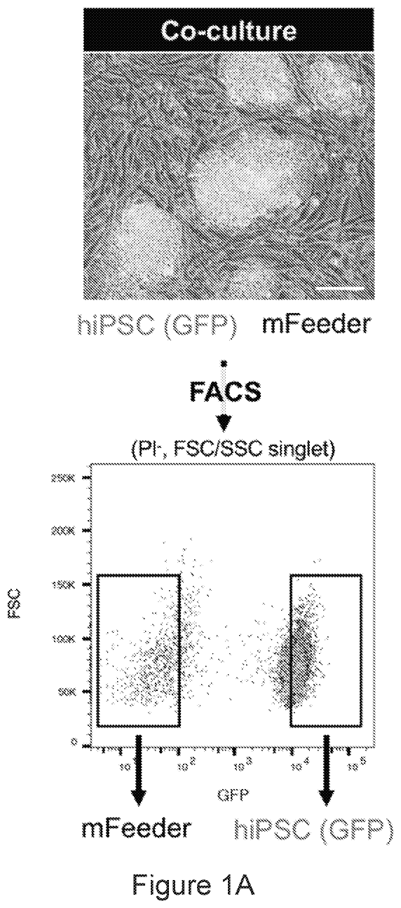

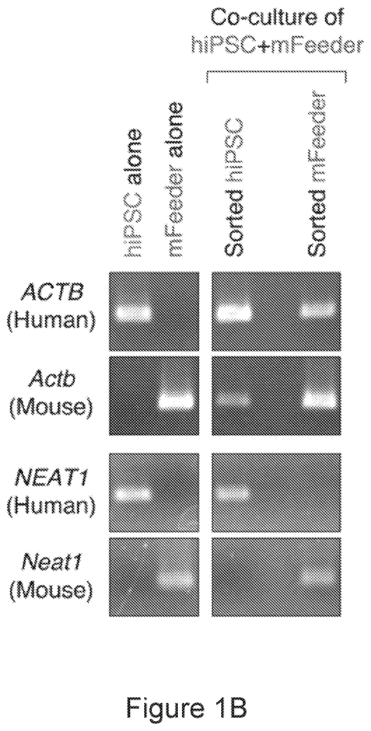

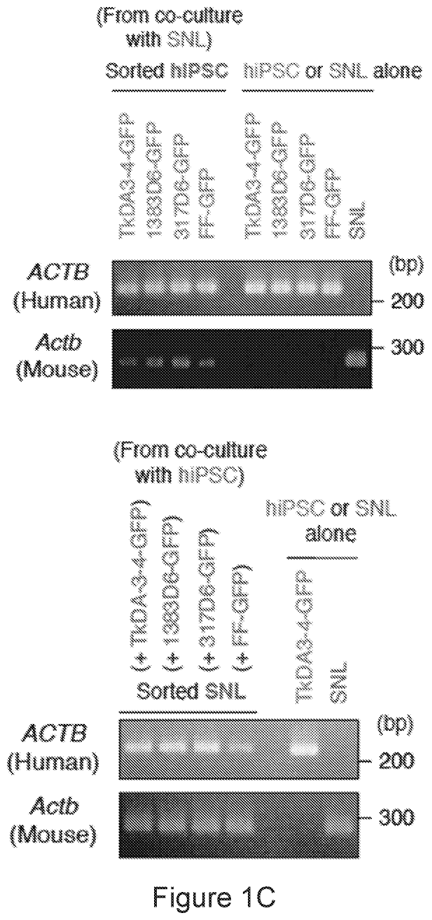

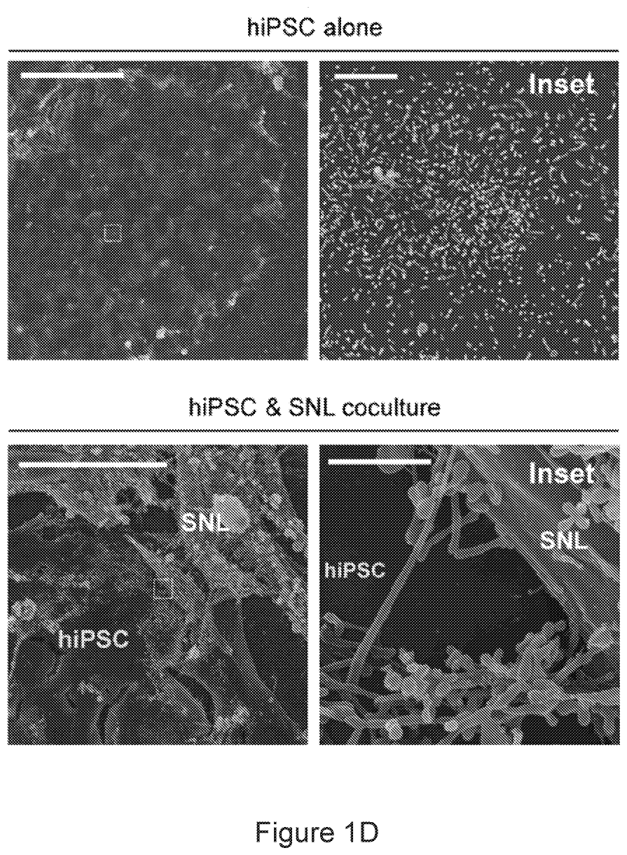

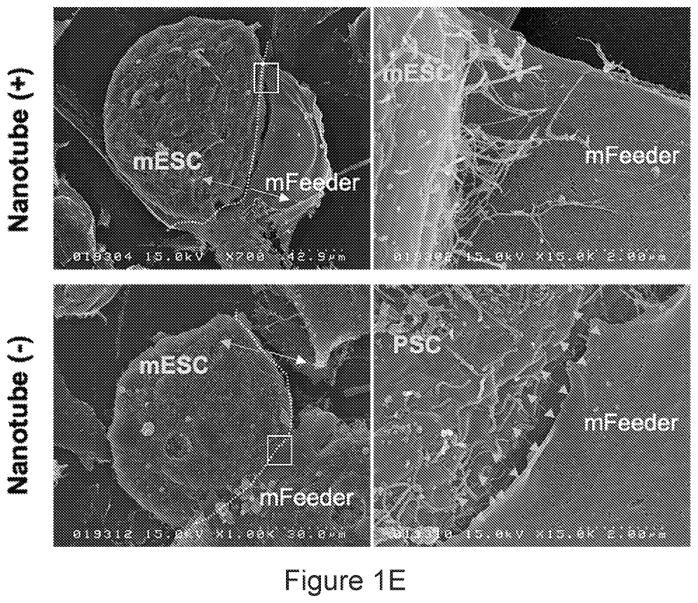

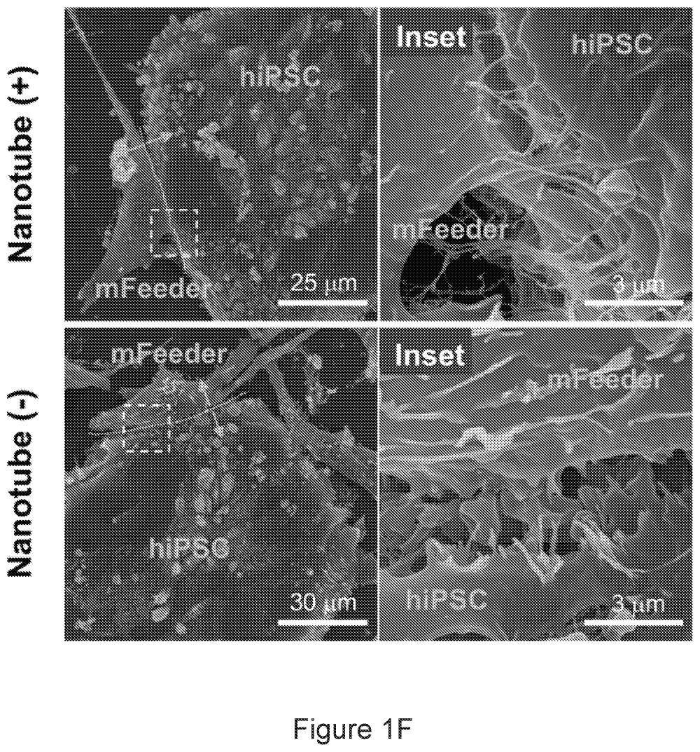

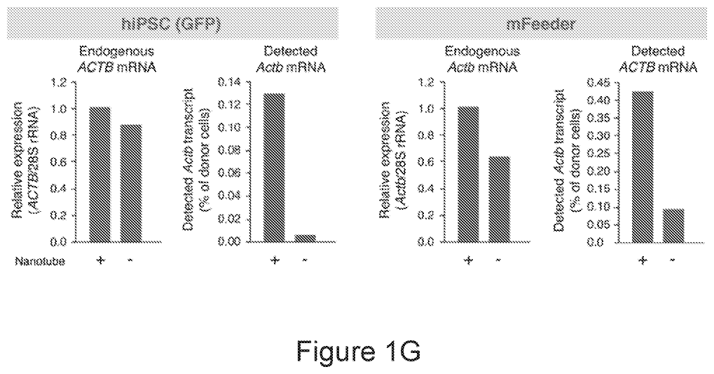

Human induced pluripotent stem cells (hiPSCs) and the mouse feeder cell line SNL 76/7 were co-cultured (FIG. 1A). After co-culture, mouse mRNAs were reproducibly detected in purified human cells after direct co-culture, and vice versa. RT-PCR of sorted cell fractions with human/mouse-specific primers revealed that mouse-specific β-actin mRNA (Actb) was detected in sorted hiPSCs whereas human β-actin mRNA (ACTB) was detected in sorted mouse feeder cells (FIG. 1B). These patterns were confirmed in all of four tested hiPSC clones transgenically engineered to express EGFP (TkDA3-4-EGFP [RRID: CVCL_RJ54], 1383D6-EGFP [RRID: CVCL_UP39], 317D6-EGFP [RRID: CVCL_K092], and FF-I01-EGFP) (FIG. 1C). RRID refers to Research Resource Identifiers and are unique identifiers for referencing scientific resources, accessible at, for example, through SciCrunch (available in the world wide web at scicrunch.org/resources). Importantly, not all transcripts were identified in this manner—the long, nuclear non-coding human RNA NEAT1 was not detected in mouse feeder cells (FIG. 1B), which is in agreement with a study showing inter-cellular transfer of β-actin mRNA, but not of nuclear long non-coding RNA (Haimovich, et al. 2017).

Tunneling nanotubes (TNTs) enable inter-cellular transport of mRNAs, microRNAs, proteins, and organelles. Thus, inter-cellular connections between mouse and human cells were probed using scanning electron microscopy analysis (SEM). Compared with hiPSCs showing short protrusions on their surface when cultured alone (FIG. 1D), a substantial number of nanotubes protruding from hiPSCs onto the surface of SNL cells was observed. Similar protruding structures were also observed in the interface between mouse embryonic stem cells (mESCs) and SNL cells (FIG. 1E). Inflammatory stimulus treatment, for example lipopolysaccharide (LPS), known as an inhibitor for intercellular coupling in vitro, effectively prevented nanotube connections among co-cultured cells (FIGS. 1F, 1E). Quantitative analyses revealed that a portion of mouse Actb mRNA corresponding to approximately 0.1% of the expression in SNL cells was detected in hiPSCs under normal conditions, while a negligible amount was detected in the LPS-treated cells (FIG. 1G). These data indicate that a subset of mRNAs is transferable between hiPSC and mouse feeder cells, potentially through inter-cellular nanotubes.

### Example 2

### Transcriptomic Analysis of Inter-Cellular Transfer Mediated mRNAs from Mouse to Human

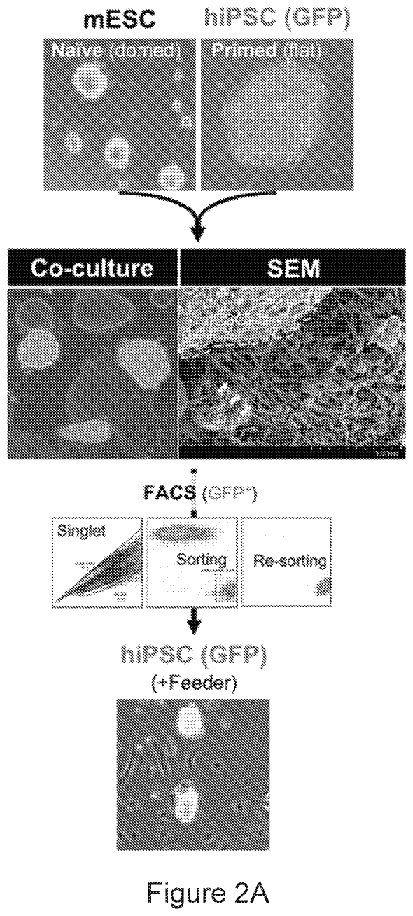

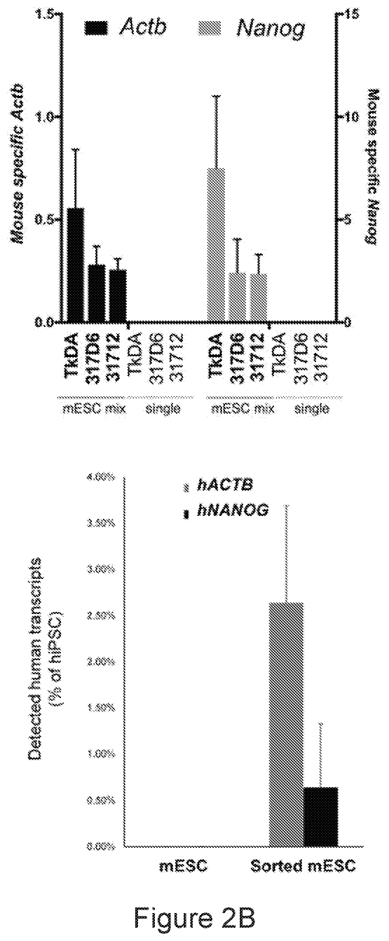

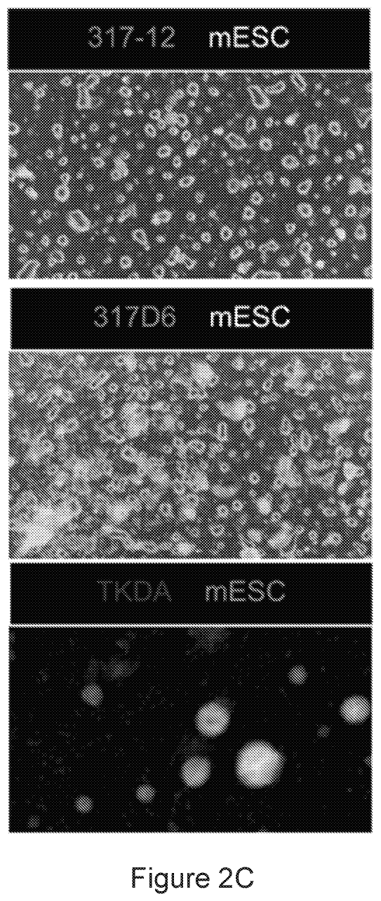

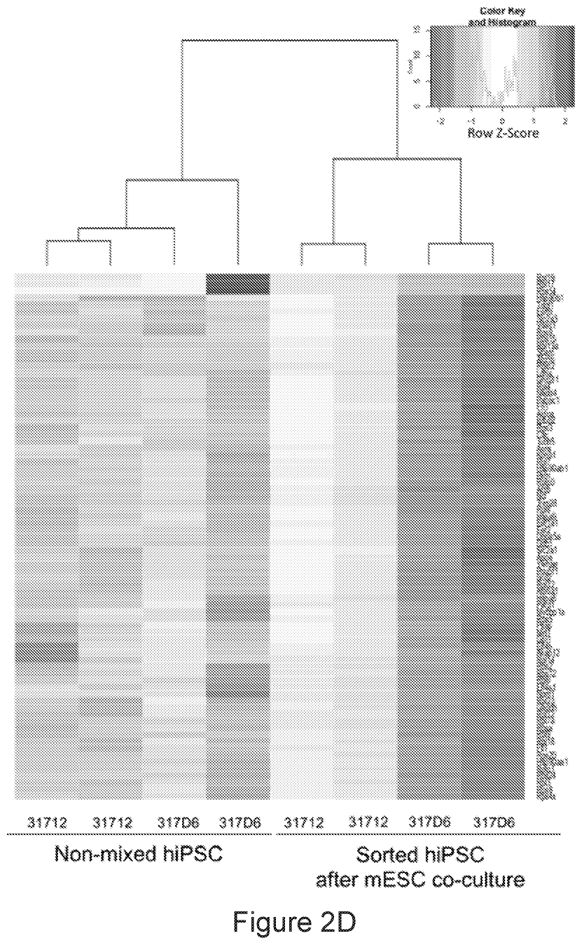

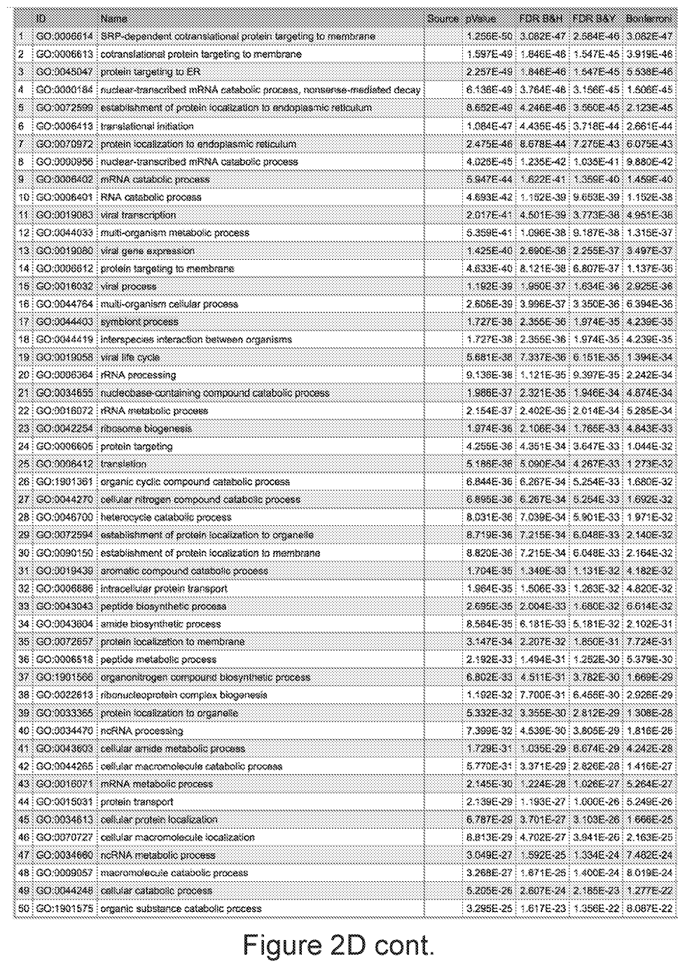

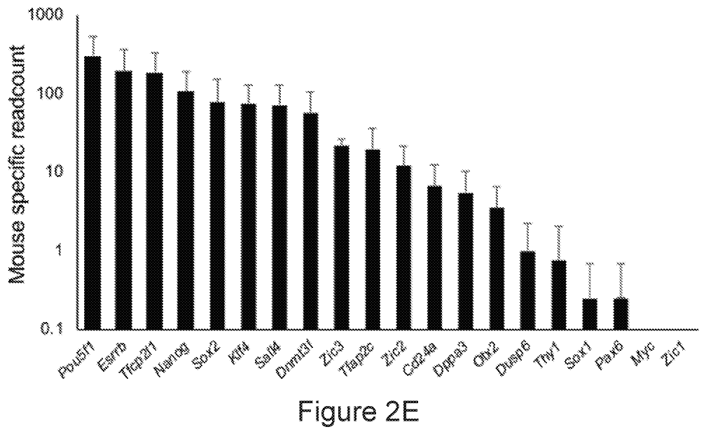

To test if this transfer occurs in other pluripotent stem cell types, hiPSCs were cultured with mESCs. mESC (naïve) and hiPSC (primed) (FIG. 2A) are in distinctive pluripotent states that require different culture media: hiPSCs are maintained in mTeSR/Matrigel conditions in a two-dimensional flat shape, while mESCs are maintained under 2i/gelatin culture with a three-dimensional colony shape. When hiPSCs are cultured under 2i/gelatin conditions, cells are unable to maintain growth and are quickly eliminated. However, hiPSCs were found to adapt into 2i/gelatin culture and grow sufficiently when co-cultured with mESCs (FIG. 2A). To examine if the mRNA transfer phenomenon contributed to this unique adaptation, mRNA expression levels in hiPSCs were evaluated after two rounds of flow-cytometric separation from mESCs based on the use of two distinct fluorescent reporter lines to minimize mouse cell contamination and/or fusion (FIG. 2A). The mouse-specific Actb mRNA was again identified in hiPSC-derived transcripts after co-culture but not in mono-culture (FIG. 2B). Conversely, mESC contained human-specific ACTB mRNA (FIG. 2B). The amount of mouse Actb and Nanog mRNA detected in hiPSC co-cultured with mESC is similar to that observed for hiPSC and SNL co-culture (FIG. 2B). All hiPSC clones (317-12, 317D6, TkDA3-4) experienced similar cell morphological conversion in the presence of mESC (FIG. 2C) as well as mouse Actb and Nanog mRNA transfer (FIG. 2B), confirming the consistency of the mRNA transfer mechanism across multiple hiPSC lines.

Next, the identity of the transfer-prone mRNA was determined by RNA-seq and subsequent separation of human and mouse sequences in the obtained reads. Co-culture with mESCs increased the abundance of mouse RNAs found within hiPSCs, with the percentage of mouse reads found in the hiPSC clones 317-12 and 317D6 relative to the total reads increasing from 0.05% (6817 mouse reads) and 0.07% (8186 mouse reads), to 0.3% (44,176 mouse reads) and 2.8% (311,859 mouse reads), respectively. The average number of unique mouse genes expressed per sample was almost three-fold higher in the co-cultured samples: 9953 genes vs 3571 for the monocultured cells. Among the top 75 genes with the highest gene expression variance, all had higher expression in the co-cultured cell lines (FIG. 2D). Gene ontology (GO) enrichment analysis revealed that many of these genes are involved in protein targeting to membrane, symbiont process, translational initiation, and RNA processing and localization. Among the mouse genes detected in co-cultured samples, 491 mouse-derived, naïve state-related transcription factor (TF) mRNAs were found in sorted hiPSCs after co-culture with mESCs (FIG. 2E). In contrast, some of the mouse-derived RNAs which are known to be highly expressed in mESCs, including Wnt1, Wnt5a and Hoxa11, were not detected while Fgfl3 only had 1 read among all samples, indicating that the transfer-competency varies among different mRNAs.

### Example 3

### Mouse ESC Derived mRNA Transfer Enables Naïve Conversion of Human iPSC

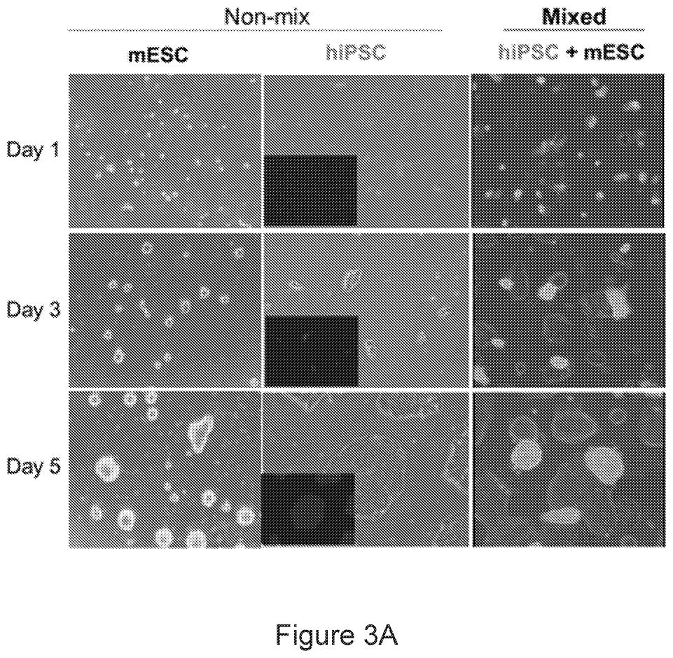

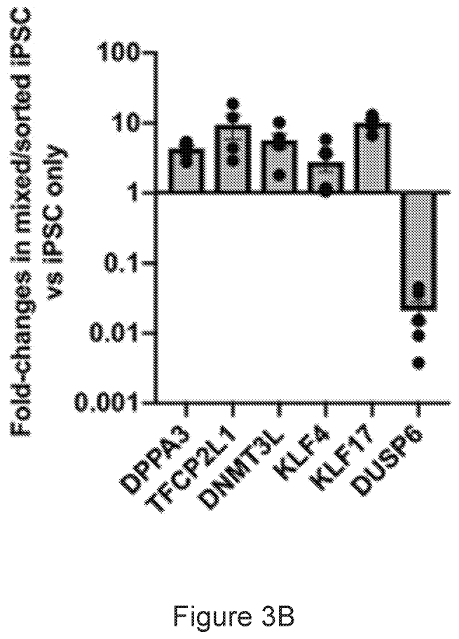

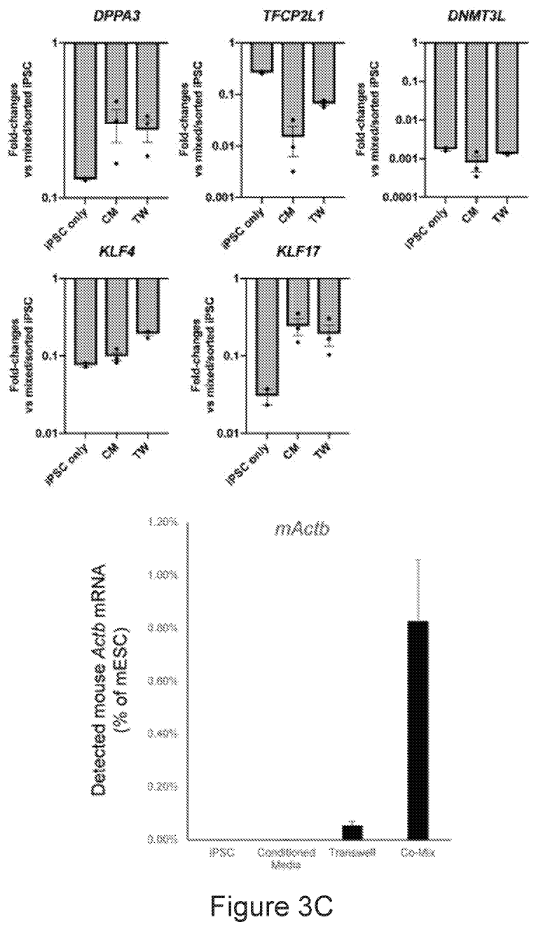

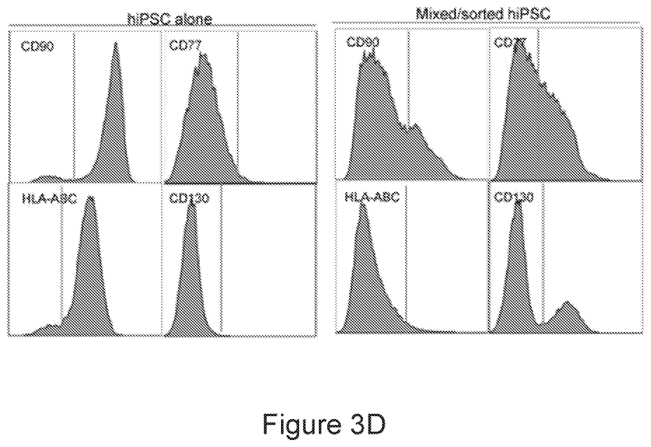

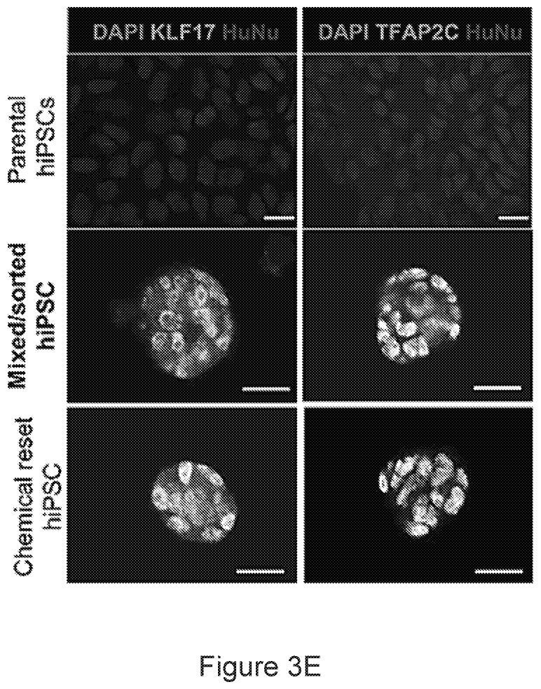

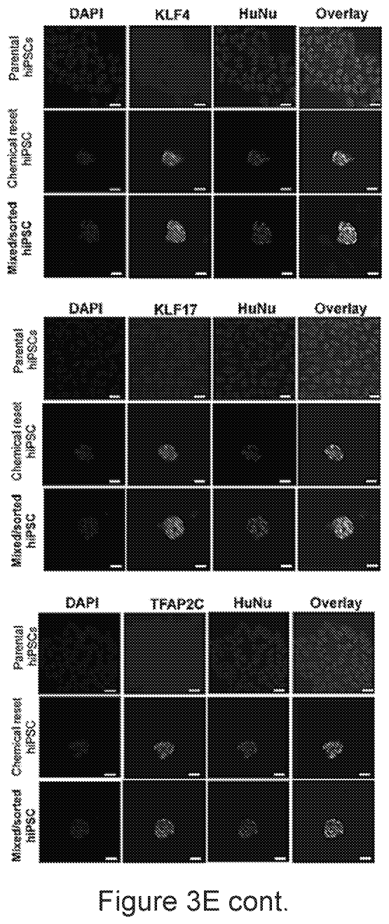

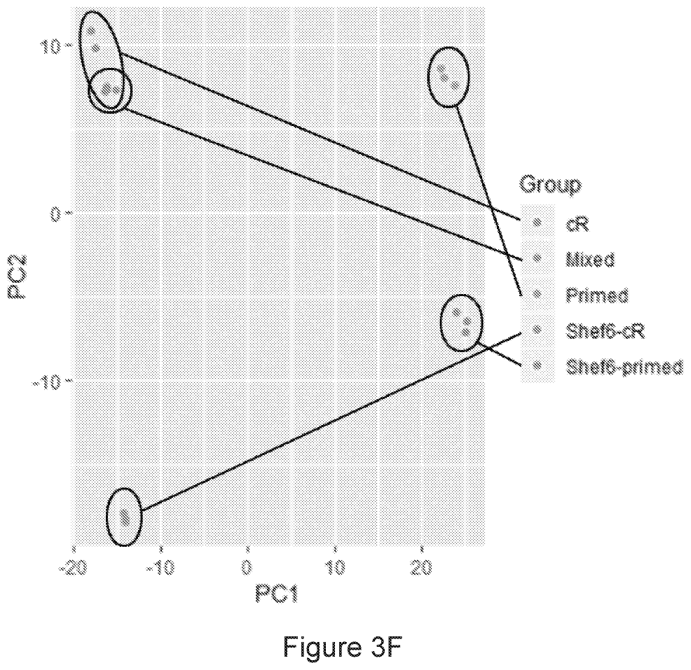

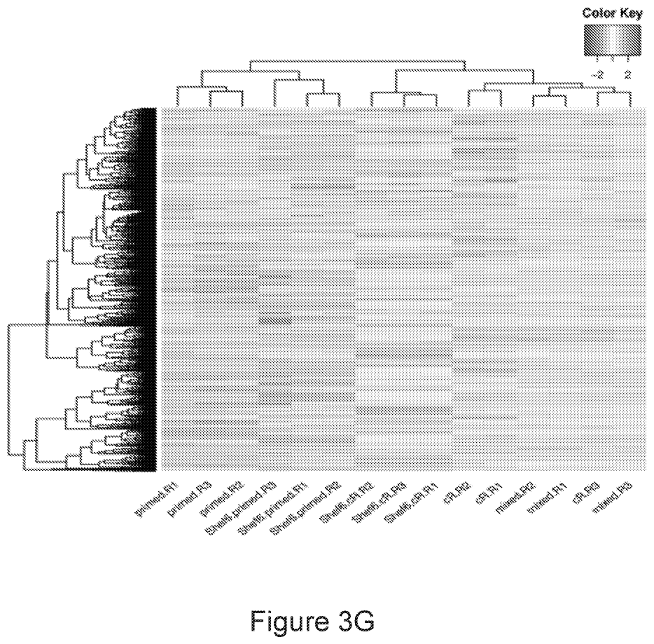

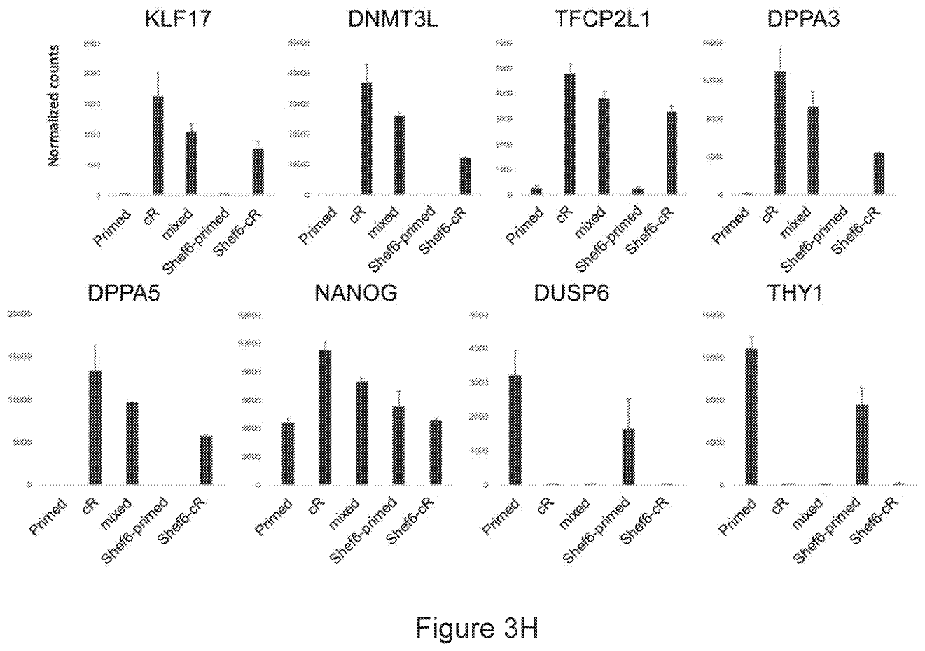

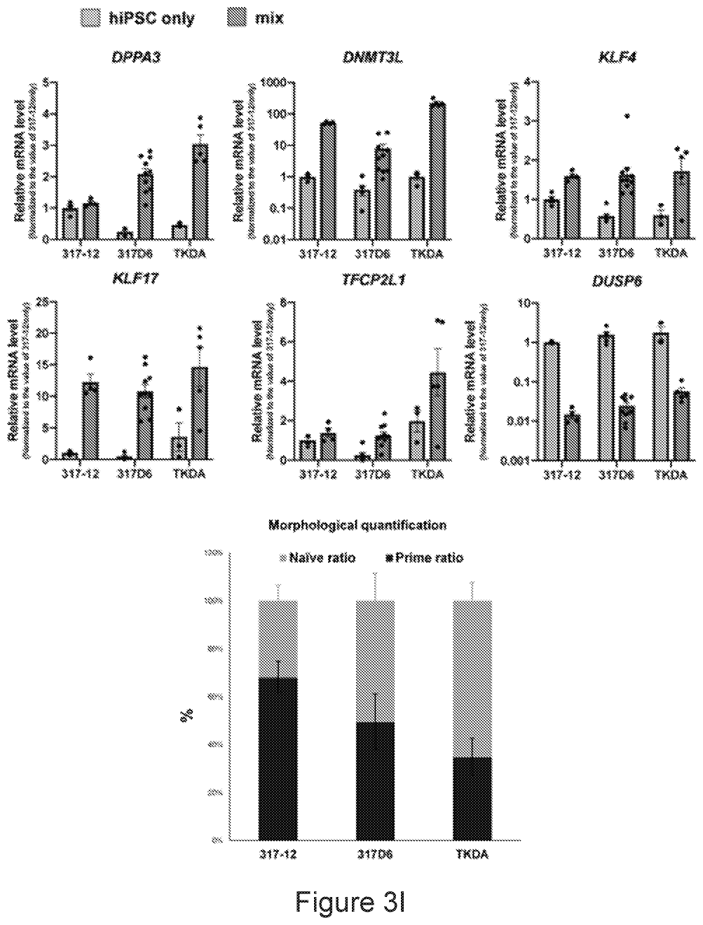

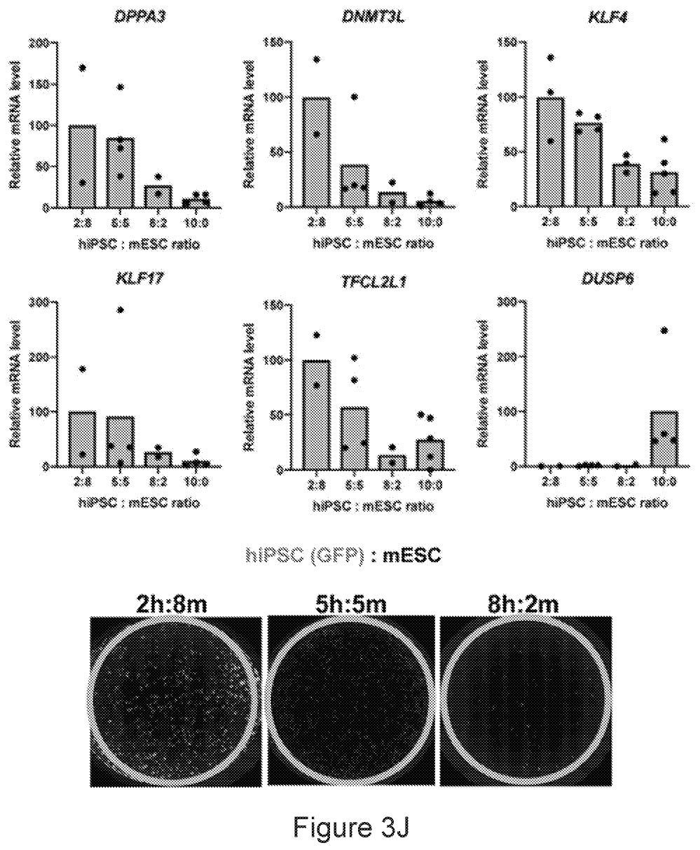

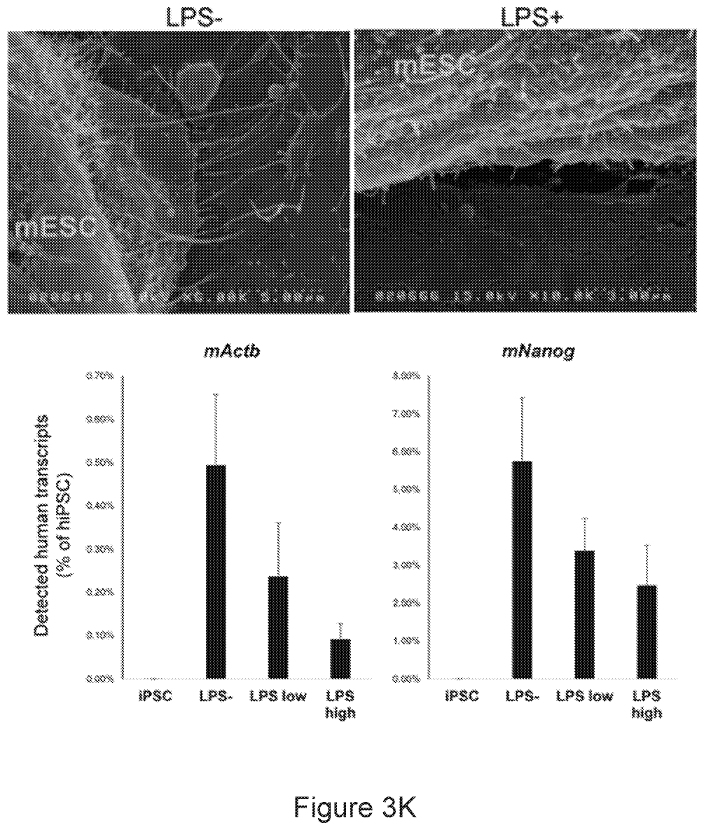

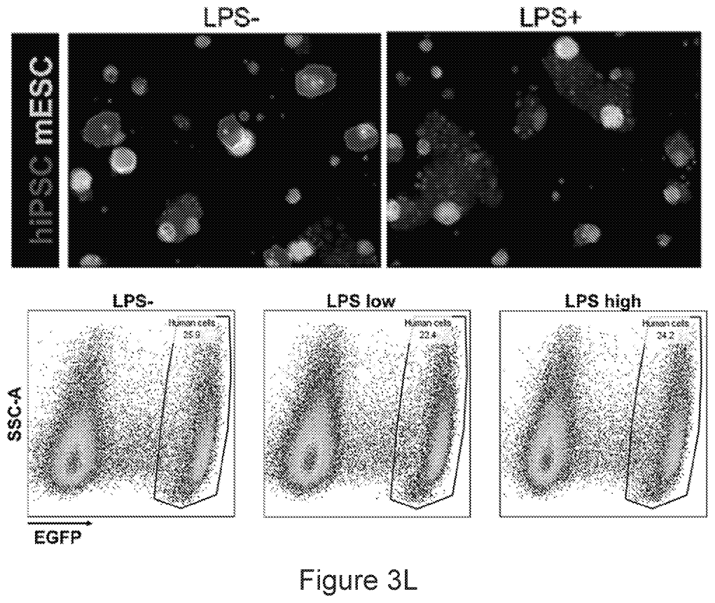

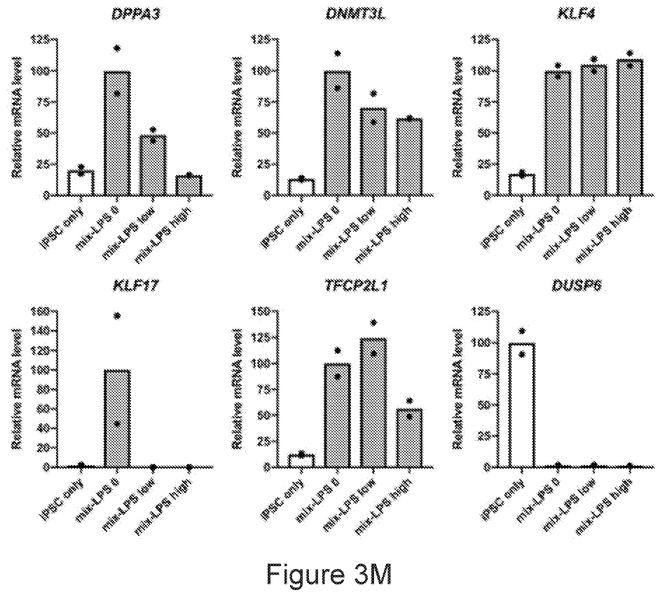

The presence of numerous naïve state mouse TF mRNAs raised the possibility that the adaptation to growth in 2i/gelatin condition involved reprogramming of hiPSCs into a naïve pluripotent state by mouse-derived TFs. After morphological conversion of hiPSCs around day 3 to day 5 (FIG. 3A), quantitative analyses of the sorted hiPSCs revealed robust expression of core naïve pluripotency markers (DPPA3, TFCP2L1, DNMT3L, KLF4 and KLF17) and down regulation of primed marker (DUSP6) (FIG. 3B). Conditioned medium from mESCs and transwell co-culture assays could not induce dome-shaped hiPSCs and resulted in negligible induction of naïve pluripotency markers with no appreciable mouse specific Actb mRNA (FIG. 3C), suggesting that naïve-related mRNA induction in hiPSCs requires direct cell to cell contact.

Flow cytometry analyses with multiplexed naïve specific and primed specific antibodies revealed that hiPSCs alone highly expressed primed-specific markers CD90 and HLAABC and did not express naïve-specific markers CD130 and CD77. Conversely, putative naïve hiPSC populations expressed naïve-specific markers CD130 and CD77 along with downregulation of primed-specific markers 10 days after co-culture with mESCs and subsequent sorting (FIG. 3D). Purified human naïve marker-expressing cells can be repeatedly propagated in human naïve maintenance medium (PXGL. KLF17 and TFAP2C are established human/primate-specific naïve pluripotency regulatory factors. To confirm full phenotypic conversion into a naïve state after passage, immunofluorescence analysis in propagated putative naïve hiPSCs was performed. Similar to chemical reset cells described in Guo, et al. (2017), hiPSCs experiencing co-culture with mESCs expressed both KLF17 and TFAP2C with human specific nuclear antigen, whereas parental non-mixed hiPSCs did not (FIG. 3E). RNA-seq was also performed for converted cells via mixed culture (mixed), chemical reset cells (cR), and those parental iPSCs to compare with originally reported naïve human PSCs (FIGS. 3F, 3G). Principal component analyses (PCA) and hierarchical clustering were performed based on genes differentially expressed between naïve and conventional PSC. In PCA, mixed/cR cells were separated from parental primed PSCs in PC1 (explains 57% variance), although there were still difference between the samples and the deposit dataset in PC2 (explains 18% variance) (FIG. 3F). In the datasets, naïve markers KLF7, DNMT3L, TFCP2L1, DPPA3, and DPPA5 were only or highly expressed in mixed/cR cells; primed markers DUSP6 and THY1 were only expressed in parental primed PSCs; whereas commonly expressing gene NANOG expression did not vary compared to those markers (FIG. 3H). Importantly, gene expression profile of inhouse cR cells and mixed cells are indistinguishable, indicating that co-cultured primed stem cells are reprogrammed to a naïve state without the need for the chemical compounds needed for cR cells. The co-culture methods were also applied to three different hiPSC lines (TkDA3-4, 317-12, and 317D6), which revealed that all of lines exhibited robust induction of naïve-like characteristics (FIG. 3I). Interestingly, a positive correlation between the conversion efficiency and hiPSC/mESC ratio was found: the greater the relative number of mESCs, the higher naïve marker gene expression in hiPSCs (FIG. 3J). These results indicate that the naïve reprogramming is reproducible and potentially promoted by direct co-culture with mESCs.

Given that nanotube connections are impaired by LPS in the co-culture models of hiPSCs/mESCs and feeder cells (Example 1), the effect of LPS on naïve-like conversion of hiPSCs was examined. Similar to the previous Example, LPS treatment ablated nanotube formation between hiPSCs and mESCs and the transfer of mouse-specific Actb and Nanog mRNAs into hiPSCs (FIG. 3K). Consistent with these results, a greater number of flat-shaped hiPSCs emerged in co-cultured condition under LPS treatment (FIG. 3L), followed by down regulation of naïve-related genes (FIG. 3M). Collectively, these data indicate that snRNA transfer-mediated reprogramming requires direct cell-to-cell contact, potentially via nanotubes.

### Example 4

### Mouse ESC Co-Culture Induces Dynamic Changes in Accessible Chromatin in hiPSC

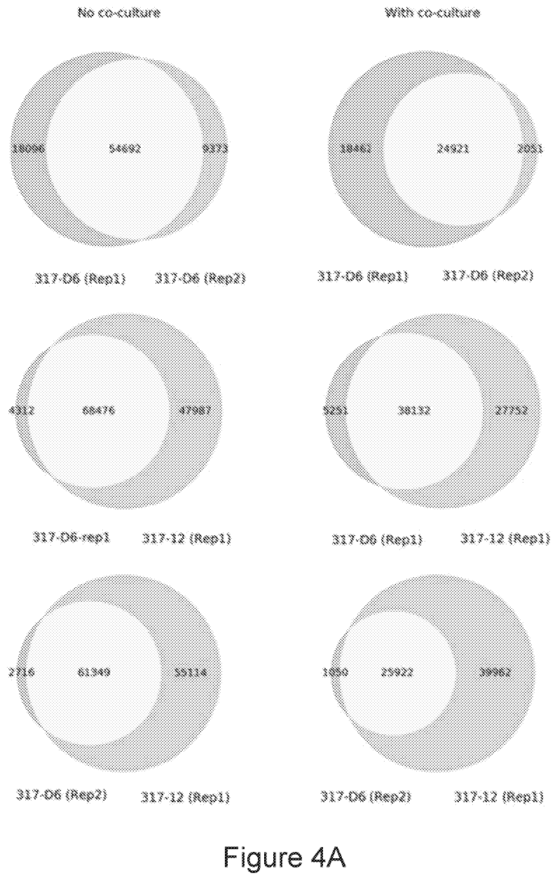

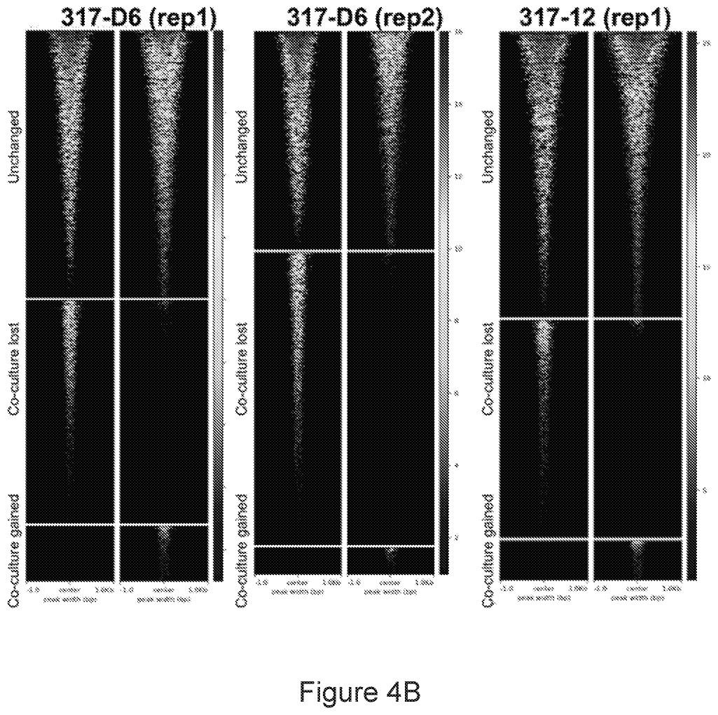

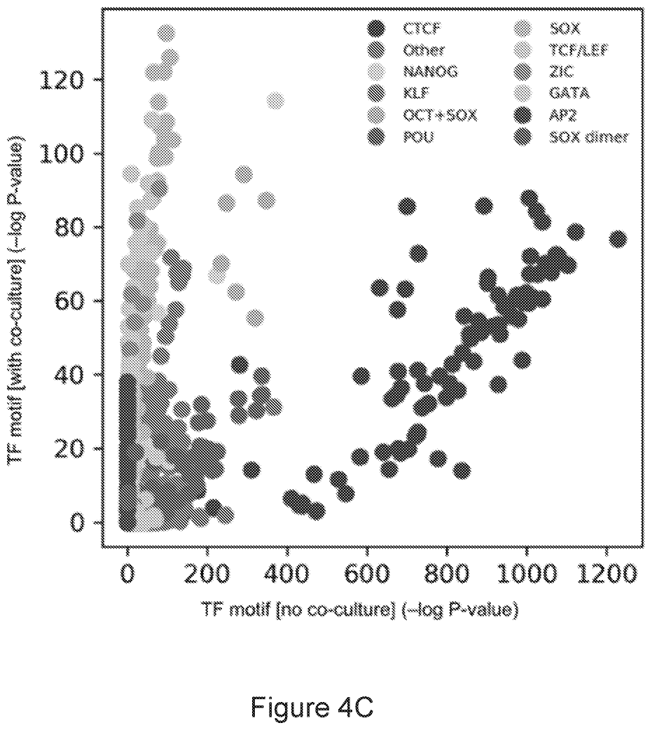

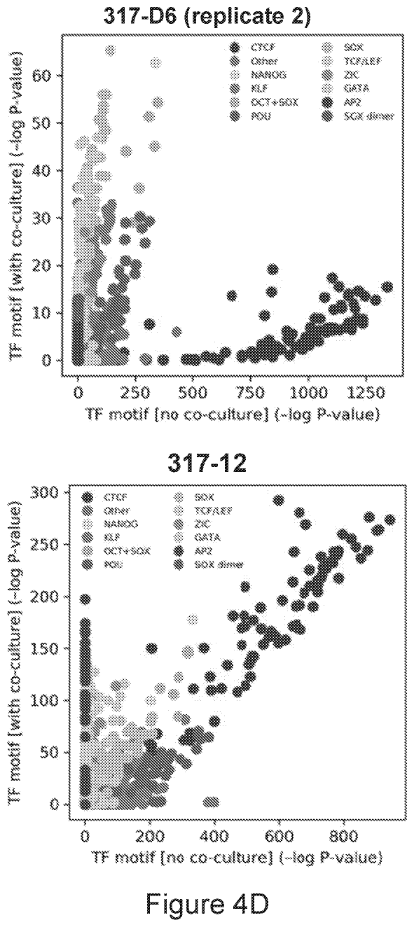

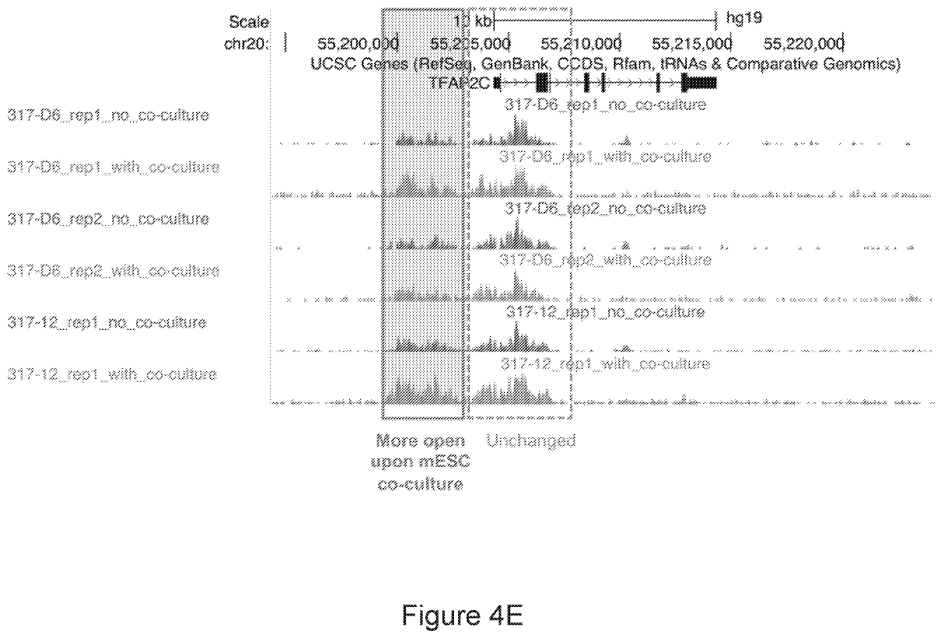

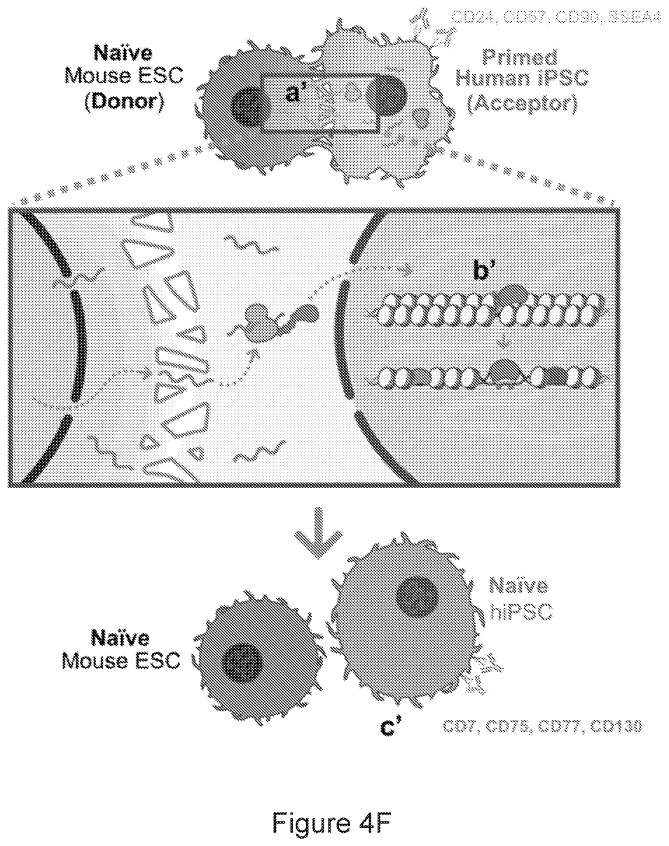

To reveal if mESC co-culture had global effects on the chromatin state of hiPSCs, ATAC-seq experiments were conducted in hiPSCs before and after co-culture, using 317-12. cells and two biological replicates of 317-D6 cells. For all three experiments, strong agreement between the resulting ATAC-seq peaks was seen (FIG. 4A). Next, regions of open chromatin that changed in the presence of the co-cultured mouse cells was identified. Over half of the total open chromatin regions showed significantly altered accessibility subsequent to co-culture. For example, of the 59,360 total ATAC-seq peaks identified in experimental replicate 1 of the 317-D6 cells, 29,079 (49%) did not significantly change between conditions (“Unchanged”), 24,307 (41%) had significantly less ATAC-seq signal in the presence of mouse cells (“Co-culture lost”), and 5,974 (10%) had significantly more signal (“Co-culture gained”), with similar proportions observed in the other two experiments (FIG. 4B).

To identify the particular TFs that might correspond to these large-scale chromatin accessibility changes, TF binding site motif enrichment analysis on the ATAC-seq peaks sets corresponding to each of these three categories was performed. Strikingly, a highly significant motif enrichment was observed for many of the same TFs whose mRNA was transferred from mESCs. For example, in 317-D6 (replicate 1) “Co-culture gained” peaks, binding motifs for SOX2 and TFAP2C were highly enriched (FIG. 4C) and had high mouse-specific gene expression detected in hiPSCs when co-cultured with mESCs (Example 2). These results are highly consistent across the three different cell types, with the same set of TFs identified in the 317-D6 (replicate 2) and 317-12 experiments (FIG. 4D) and are also highly consistent with the enriched motifs previously reported. Notably, many of the regions identified are located proximal to biologically important genes. For example, highly reproducible “Co-culture gained” peaks are located immediately upstream of the TFAP2C promoter (FIG. 4E). Taken together, these data indicate that a portion of TF mRNAs are transferred from naïve mESCs (“influencer”) into the neighboring primed hiPSCs (“recipient” or “acceptor”), and then chromatin reorganization at the corresponding IF binding loci is induced to reprogram hiPSCs to naïve-like pluripotent state (FIG. 4F).

MicroRNAs and incomplete mRNAs undergo transfer via extracellular vesicles (e.g. exosomes). In contrast to this diffusion-based transfer mechanism, reports using immortalized cells or fibroblasts suggest that full-length mRNAs can also undergo direct cell-cell transfer via cytoplasmic extensions characteristic of membrane nanotubes, which connect influencer and acceptor cells. Without being limited by any mechanism of action, described herein is evidence that mRNAs including those encoding pioneer transcription factors are transferable between contacting cells, resulting in alterations of the acceptor cell transcriptome and epigenome.

### Example 5

### Silencing of Mouse Tfcp2l1 and Tfap2c in Human iPSC Inhibits their Naïve Conversion Induced after Co-Culture with Mouse ESC

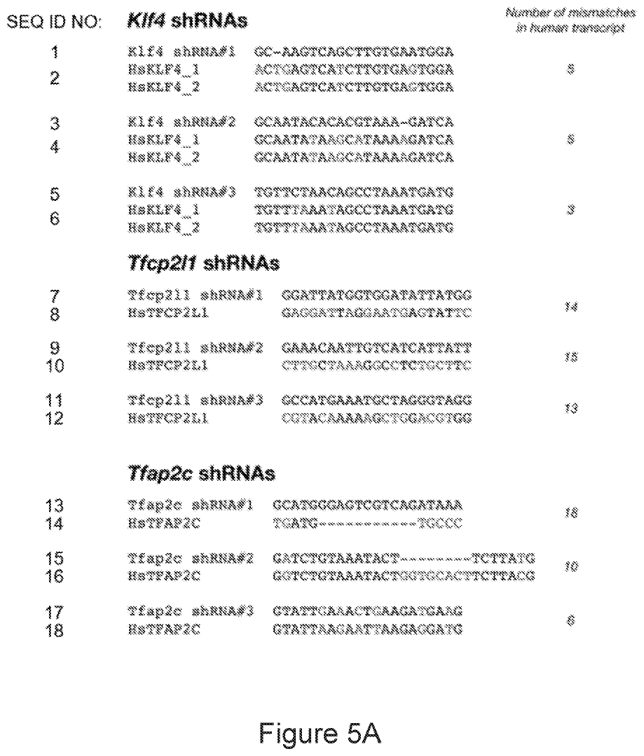

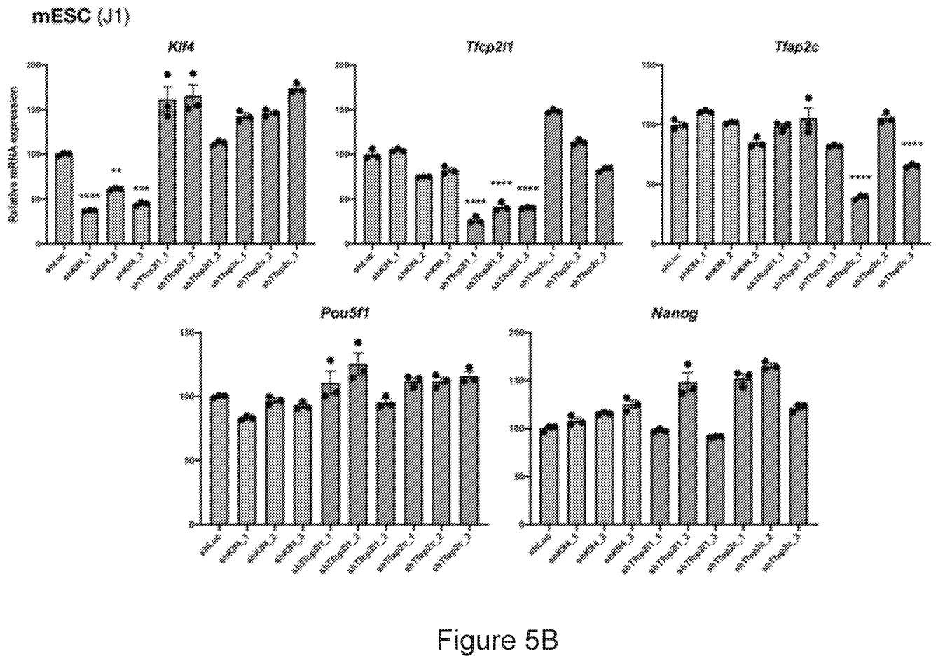

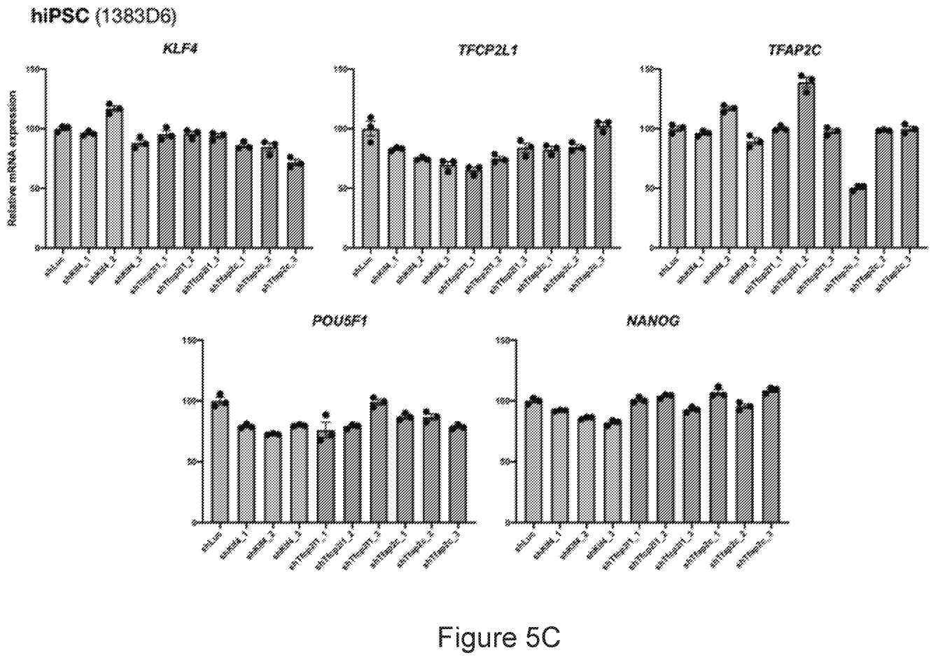

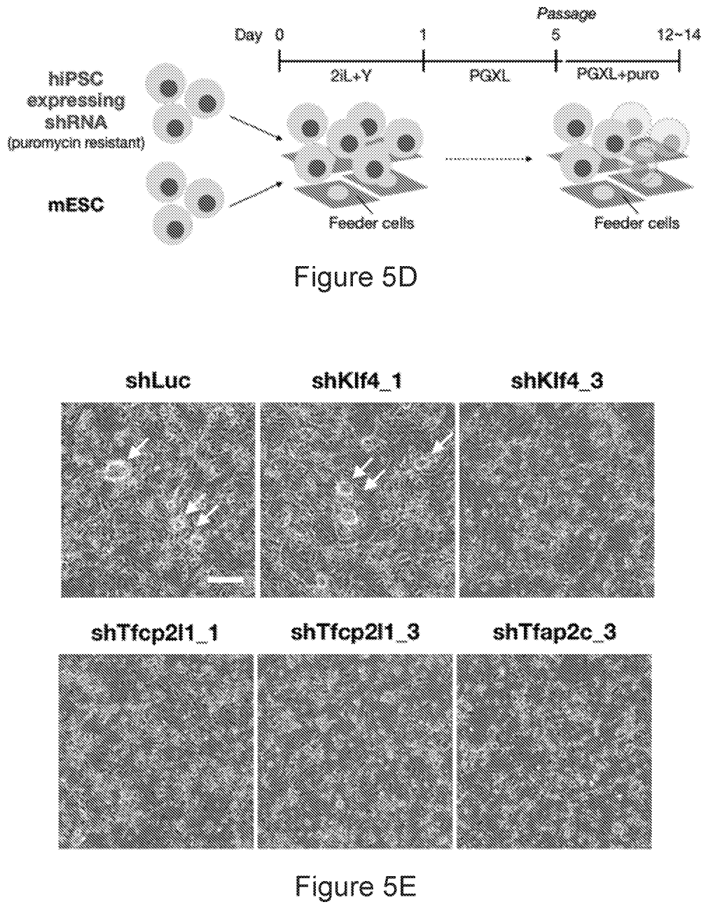

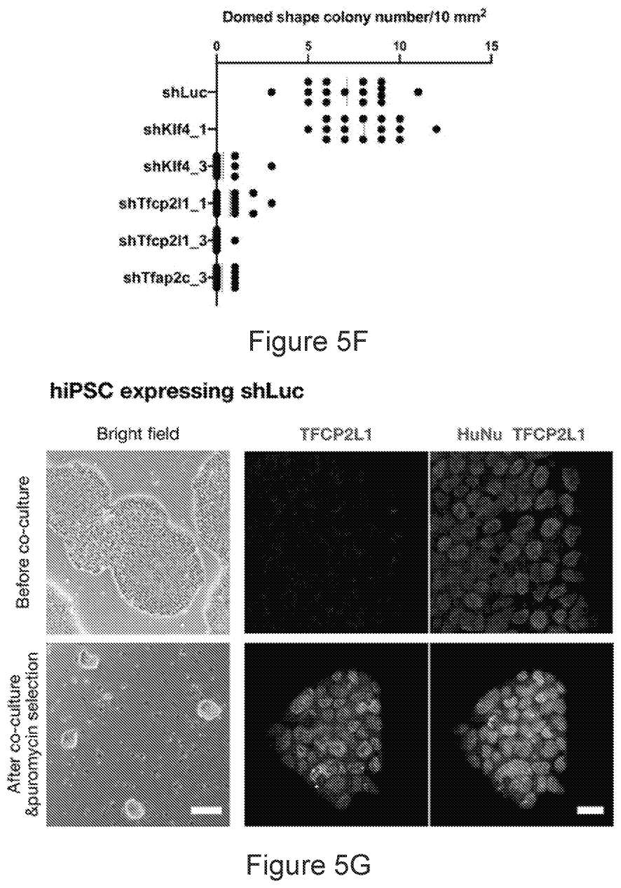

To further confirm that the transferred mRNA from the mouse donor ESCs were reprogramming the recipient human primed iPSCs to a naïve state, shRNAs specific for mouse Klf4, Tfcp2l1, and Tfap2c were designed. The sequences for the shRNAs were designed within regions of divergence between mouse and human genomic sequences, to avoid off-target effects with native human KLF4, TFCP2L1, and TFAP2C (FIG. 5A). Efficacy of the designed shRNA were first demonstrated in mouse ESCs. Viral shRNA vectors were prepared and used to infect mouse ESCs, and expression of Klf4, Tfcp2l1, Tfap2c, Pou5f1, and Nanog was assessed by qRT-PCR. Significant downregulation of expression of the corresponding genes is observed for the three transcription factor-targeting sHRNAs, whereas expression of Pou5f1 and Nanog are not affected (FIG. 5B). Conversely, human iPSCs infected with the shRNA vectors did not exhibit downregulation of any gene, showing that the mouse-specific shRNAs were ineffective against human orthologues (FIG. 5C). FIG. 5D depicts the experimental design for assessing the effect of shRNA silencing on naïve reprogramming. Briefly, puromycin resistant hiPSCs expressing the mouse-specific shRNA and puromycin-sensitive mESCs are co-cultured to induce reprogramming of the hiPSCs to the naïve state. After 5 days of co-culture, puromycin is added to the media to select for the hiPSCs. Out of the shRNAs tested (Klf4 #1, Klf4 #3, Tfcp2l1 #1, Tfcp2l1 #3, and Tfap2c #3), the number of domed colonies indicating naïve state hiPSCs were greatly reduced in Klf4 #3, Tfcp2l1 #1, Tfcp2l1 #3, and Tfap2c #3 shRNA conditions (FIGS. 5E-F). In control hiPSCs (expressing Luciferase-specific shRNA [shLuc]), TFCP2L1 expression is observed after co-culture with mESCs and puromycin selection (FIG. 5G-). This demonstrates that silencing of transferred mouse naïve transcription factors reduces efficacy of reprogramming of primed hiPSCs to a naïve state.

### Example 6

### Expression of Mouse Derived Transcription Factor Proteins in Human iPSC Induced after Co-Culture with Mouse ESC

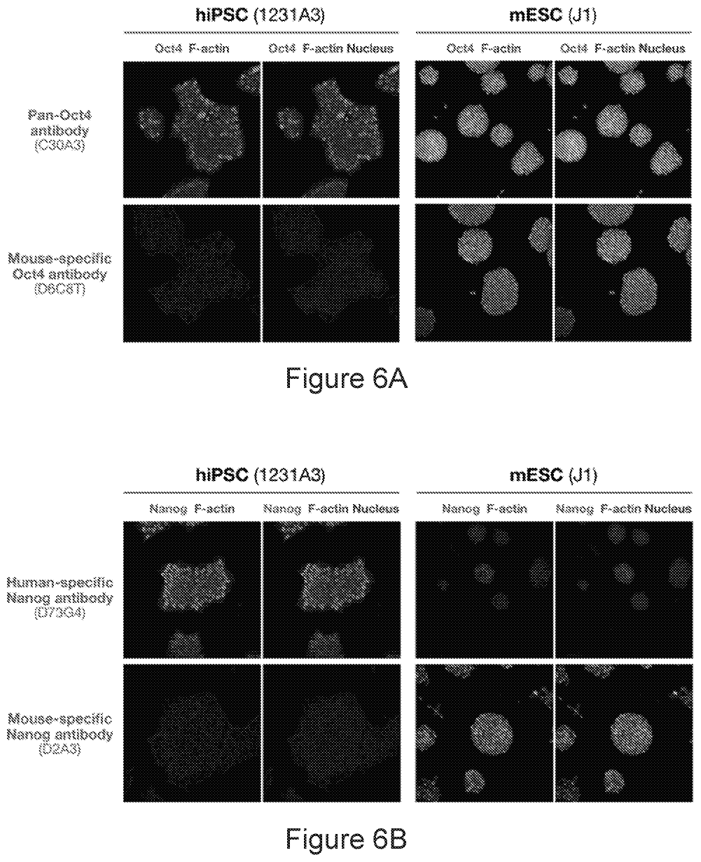

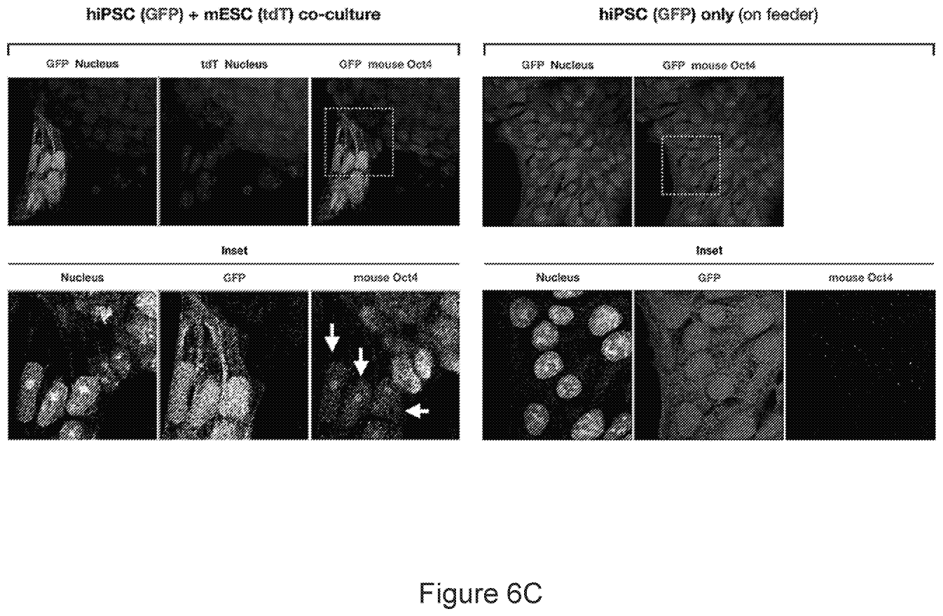

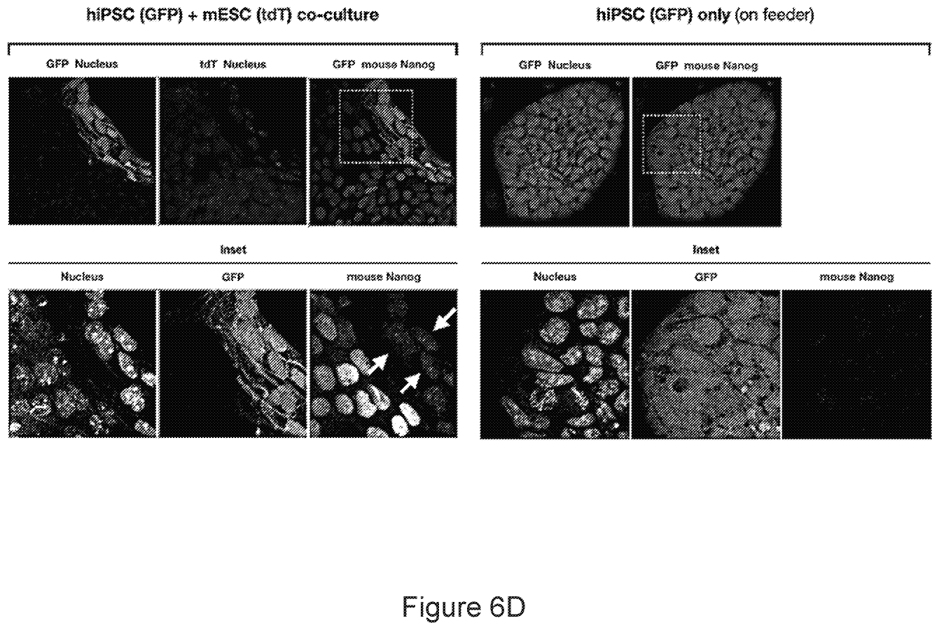

Expression of mouse transcription factor proteins following co-culture was investigated. hiPSCs and mESCs were stained with pan- and mouse-specific Oct4 (FIG. 6A) and human- and mouse-specific Nanog (FIG. 6B) antibodies. While the pan-Oct4 and human-specific Nanog antibodies strongly labeled the hiPSCs, these cells did not exhibit cross-reactivity with the mouse-specific Oct4 and Nanog antibodies. After co-culture of hiPSC expressing GFP and mESC expressing tdTomato, the hiPSCs showed weak reactivity, relative to the adjacent mESCs, for the mouse-specific Oct4 and Nanog antibodies, suggesting that inter-cellular transfer of mRNA from the mESCs is resulting in low levels of mouse-specific protein expression in hiPSCs (FIGS. 6C-D).

### Example 7

### Materials and Methods

Cell Culture: SNL 76/7 feeder cells were cultured in Dulbecco's modified Eagle's medium (DMEM) supplemented with 10% fetal bovine serum (FBS), 0.1 mM MEM non-essential amino acids, and 2 mM L-glutamine.

Primed human iPSCs (hiPSCs) were routinely cultured in mTeSR1 on Matrigel growth factor reduced (BD Biosciences)-coated plates or in StemFit (AK03N, Ajinomoto) on Laminin-511 E8 (Nippi)-coated dishes for the TKDA3-4, 317D6 and 317-12 iPSC clones. To passage, 80-90% confluent hiPSCs were dissociated with Accutase (Millipore) and then replated onto 1:30 diluted Matrigel growth factor reduced or 0.5 μg/cm² Laminin-511 E8-coated 6-well tissue culture plates (Corning) at 1×10⁵ cells per well with 10 μM ROCK inhibitor Y-27632 for 1 day and fresh media without Y-27632 the following days.

The hiPSC lines (TKDA3-4, 1383D6) stably expressing GFP were maintained on SNL feeder cells in StemFit (AK02N, Ajinomoto) to analyze inter-cellular RNA transfer. After 3-5 days, the cells were sorted using a FACSAria II cell sorter (BD Biosciences) as GFP-positive and GFP-negative cells, followed by total RNA extraction.

Naïve mouse ESCs (mESCs) were cultured in conditions of 1:1 mixture of DMEM/F12 and Neurobasal media, 1×N2-supplement, 1×B27-supplement, 2 mM Glutamine, 50 U/ml and 50 μg/ml penicillin-streptomycin (all from ThermoFisher Scientific), 1.5×10⁻⁴ M monothioglycerol (Sigma M6145), 50 μg/ml bovine serum albumin (Sigma), 10 ng/ml recombinant mouse LIF (Millipore). 3 μM CHIR99021 (Tocris), 1 μM PD0325901 (Sigma), on a MEF-layer seeded at a density of 1×10⁵ cells per 6-well plate. Cells were passaged with 5 min incubation with Accutane (ThermoFisher Scientific).

For conversion of primed hiPSCs to naïve state, naïve MESC and primed hiPSCs were dissociated into single cells with Accutase and a total of 5×10⁵ cells (2.5×10⁵ hiPSCs+2.5×10⁵ mESCs) per 12-well were plated in naïve mESC media with 10 μM Y-27632 onto MEF seeded at a density of 0.5×10⁶ cells per 12-well plate. The following day, media was changed to naïve mESC media or PXGL medium without Y-27632. Dome-shaped naïve colonies could be seen as early as five days after plating, and cells were treated with Accutase into single cells on day 5 followed by selective sorting of hiPSCs using FACS. Sorted cells were maintained in PXGL medium under 5% O₂ at 37° C., in which naïve markers could be detected 7-10 days after the co-culture. LPS was added into the mixed cells at a lower concentration of 100 ng/ml and higher concentration of 500 ng/ml during the 5 days co-culture.

PXGL medium is prepared by supplementing N2B27 medium with 1 μM PD0325901 (MEK inhibitor), 2 μM XAV939 (Wnt inhibitor), 2 μM Gö6983 (PKC inhibitor) and 10 ng/mL human LIF. N2B27 medium is prepared by mixing 487 mL of DMEM/F12 and 487 mL of Neurobasal media and supplementing with 10 mL of B2.7 supplement, 5 mL of N2 supplement, 10 mL of 200 mM L-glutamine, and 1 mL of 0.1 M β-mercaptoethanol. N2 medium. Non-commercial N2 supplement is prepared by supplementing DMEM/F12 basal medium with 0.4 mg/mL insulin, 10 mg/mL Apo-transferrin, 3 μM sodium selenite, 1.6 mg/mL putrescine, and 2 μg/ml progesterone.

Mitotic inactivation of feeder cells: To prepare the feeder cells, MEF and SNL cells reaching 90% confluency were treated with 10 μg/mL of Mitomycin C (Fujifilm Wako) for 3 hours. After extensive wash with PBS and trypsinization, the Mitomycin C-treated cells were plated at 7.5×10⁴ cells/cm² in gelatin-coated culture dishes. Feeder cell dishes were used within a week of plating. The original culture medium was changed to stem cell growth medium just before hiPSC were added.

Flow cytometry: Primed and naïve hiPSCs were dissociated into single cells with Accutase, washed and passed through 30-40 μm cell strainers. Conjugated antibodies were mixed with 50 μL PBS (BD Biosciences) and applied to 50-100 μL of cells (2-5×10⁵ cells per reaction). Cells were incubated for 30 min at 4° C. in the dark and washed twice with buffer (2% FBS in PBS) and centrifuged at 300×g for 5 min. Cells were resuspended in buffer and analyzed with a BD LSRFortessa cell analyzer (BD Biosciences) or a BD FACSAria Fusion for cell sorting. Single-stained cells or OneComp eBeads (eBioscience) were used for compensation calculations. Data was analyzed using FlowJo V10.1 software (FlowJo, LLC).

Immunofluorescent microscopy: hiPSCs were plated on Matrigel- and MEF feeder-coated Lab-TekII chamber slide (Nunc) and cultured under 5% O₂ at 37° C. for 2 days. The cells were fixed with 4% paraformaldehyde (Wako) for 10 min at room temperature (RT), and permeabilized with 0.3% Triton X-100 (Sigma) in PBS for 10 min at RT. Cells were blocked with MAXblock blocking medium (Active Motif) for 1 hour. Primary and secondary antibodies were diluted in MAXblock blocking medium, and were applied for 1 hour and 20 minutes, respectively. DNA was counterstained with 1 μg/mL DAPI (Thermo Fisher) for 15 min. The samples were washed twice with PBS between each step. Images were taken with an FV3000 confocal microscope (Olympus).

qPCR: Total RNA was extracted using the RNeasy Mini Kit (QIAGEN). 1 μg RNA was reverse transcribed using SuperScript III (Thermo Fisher) followed by quantitative PCR with Taqman universal master mix and Taqman assays (Thermo Fisher) using the StepOnePlus Real-Time PCR System (Thermo Fisher). RNA samples from three or four biological replicates were used for each condition.

RT-PCR: Total RNA from the sorted cells was extracted with TRI reagent (Molecular Research Center, Inc.). First-strand cDNA was synthesized with PrimeScript RT reagent Kit with gDNA Eraser (Takara). RT-PCR was performed with Tks Gflex DNA Polymerase (Takara) and specific primers described below. Quantitative PCR was performed with Thunderbird SYBR qPCR Mix (Toyobo) on an ABI StepOnePlus Real Time PCR System (Applied Biosystems). To normalize the relative expression, a standard curve was prepared for each gene for relative quantification, and the expression level of each gene was normalized to the ribosomal 28S RNA genes.

Scanning electron microscopy: Cells were cultured on a cell-tight C-1 cell disk LF (MS-0113K; Sumitomo Bakelite) coated with gelatin. After the indicated treatment, they were fixed in 2.5% glutaraldehyde in 0.1 M phosphate-buffer for 2 h. They were washed overnight at 4° C. in the same buffer and post-fixed with 1% OsO₄ buffered with 0.1 M phosphate-buffer for 2 h. The specimens were dehydrated in a graded series of ethanol and dried in a critical point drying apparatus (JCPD-5; JEOL) with liquid CO₂. They were spatter-coated with platinum and examined by scanning electron microscopy (S-4500; Hitachi, Tokyo, JAPAN).

RNA-sequencing (RNA-seq): Total RNA was purified using an RNeasy Micro Kit (Qiagen) according to the manufacturer's instructions. RNA quality and quantity were checked using Bioanalyzer (Agilent) and Qubit (Life Technologies) machines, respectively. The initial amplification step was performed with the NuGEN Ovation RNA-Seq System v2, which facilitates an assay that amplifies RNA samples and creates double-stranded cDNA. Libraries were then created with the Nextera XT DNA Sample Preparation Kit (Illumina) and sequenced with an Illumina HiSeq 2500 system. RNA-seq data analyses were performed using the BioWardrobe Experiment Management System [accessible on the world wide web at github.com/Barski-lab/biowardrobe]. Briefly, reads were mapped to the mm10 genome using TopHat [version 2.0.9] and assigned to RefSeq genes (which have one annotation per gene) using the BioWardrobe algorithm. PCA was performed using the top 1000 most variable genes across experimental condition. The first and second principal components were plotted. Differential gene expression analyses were performed via DESeq2 in the BioWardrobe environment. For gene ontology analyses, the Database for Annotation, Visualization and Integrated Discovery (DAVID) was used.

To separate mouse and human sequences, the function bbsplit from the bbtools suite (BBTools) was used to bin each read from the RNA-seq fastq into a separate fastq files based on whether the read was specific to the human (hg19) or mouse (mm10) genome. To create the most stringent conditions, perfect mode was set to true and all ambiguous reads were discarded. Reads that were specific to mouse within the human cells were aligned to the mm10 genome using STAR (v2.5.1b). The resulting aligned sam file was sorted and converted to a barn file using the samtools sort function. Reads for each gene were counted using the R function summarizeOverlaps from the GenomicAlignments library using the counting mode of Union. DESeq2 was used to quantify differential gene expression among samples. To compare gene expression profile of established human naïve cell lines with originally reported naïve PSC lines, deposited sequencing data of Shef6-primed (accession numbers: ERR1924246, ERR1924247, ERR1924248.) and Shef6-cR (accession numbers: ERR1924234, ERR1924235, ERRI 924236.) were obtained from the European Nucleotide Archive. The sequencing data were uploaded to the Galaxy web platform, and we used the public server at usegalaxy.org to analyze the data. Adapter sequences were removed with Trimmomatic Galaxy Version 0.36.5 on Galaxy server. Transcript abundances were quantified using Salmon Galaxy Version 0.11.2. Transcript abundances were converted to count data with Bioconductor package tximport 1.12.0 (Soneson, Love, and Robinson F1000Res 2015), and normalized with Bioconductor package DESeq2 1.24.0 followed by summarization to gene level. Gene annotation for Homo sapiens (GRCh38) was obtained from Ensemble. Principal component analysis was performed for genes differentially expressed in naïve and primed PSCs by prcomp function based on log2-transformed normalized count value computed with scale function of R 3.6.0. Euclidian distance ware estimated based on log2-transformed normalized counts, and cluster analysis was performed for the same genes with heatmap.2 function of gplots 3.0.1.1.

ATAC-seq library preparation and sequencing: ATAC-seq libraries were prepared. Approximately 1,000,000 primed human iPSCs and naïve human PSCs were used. Briefly, samples were lysed in 50 μL of lysis buffer (10 mM Tris-HCl (pH 7.4), 10 mM NaCl, 3 mM MgCl2 and 0.1% NP-40). Immediately after lysis, nuclei were spun at 500×g for 5 min to remove the supernatant. Nuclei were then incubated with Tn5 transposase and tagmentation buffer (Illumina) at 37° C. for 30 min. After tagmentation, the transposed DNA was purified with a MinElute kit (Qiagen). Polymerase chain reaction (PCR) was performed to amplify the library using the following conditions: 72° C. for 5 min; 98° C. for 30 s; thermocycling at 98° C. for 10 s, 63° C. for 30 s and 72° C. for 1 min; and 72° C. for 5 min as the final elongation. qPCR was used to estimate the number of additional cycles needed to generate products at 25% saturation. Typically, two to five additional PCR cycles were added to the initial set of five cycles. The library was purified by AMPure XP beads (Beckman). Size selection of library pools was achieved by agarose gel electrophoresis, excising gel slices in the 250- to 500-bp range. Pools purified from gel slices were analyzed on an Agilent Bioanalyzer, and 75-bp single-read sequencing was performed using an Illumina HiSeq 2500 platform per standard operating procedures.

ATAC-seq data analysis: FASTQ files from ATAC-seq experiments were analyzed using the MARIO next generation sequencing pipeline. Briefly, QC is performed using FastQC (v0.11.2) and Trim Galore (v0.4.2), a wrapper script that calls cutadapt (v1.8.1), is used to remove adapter sequences. Reads are then aligned to the genome using bowtie2 (v2.3.4.1) with the settings “−D 15 —R 2 −L 22 −i S,1,1.15—score-min L,−0.6,−0.6, −N 0”. To account for possible cell type contamination in the experiments, any reads that aligned to the mouse genome mm9) were first removed. The remaining reads were then aligned to the reference human genome (hg19/GRCh37). The hg1.9 aligned reads (in .BAM format) were then sorted using samtools (v1.8.0) and duplicate reads were removed using picard (v1.89) using the parameters

“MAX_SEQUENCES_FOR_DISK_READ_ENDS_MAP=50000, MAX_FILE_HANDLES_FOR_READ_ENDS_MAP=8000, SORTING_COLLECTION_SIZE_RATIO=0.25, OPTICAL_DUPLICATE_PIXEL_DISTANCE=100, VALIDATION_STRINGENCY=STRICT, COMPRESSION_LEVEL=5, MAX_RECORDS_IN_RAM=500000.”

Finally, ATAC-seq peaks were called using MACS2 (v2.1.0) with parameter settings “effective genome size=2.70e+09, band width=300, model fold=[5, 50], qvalue cutoff=1.00e−02”.

To identify regions of differential chromatin accessibility between experimental conditions, MAnorm was used with default parameter settings for peak width (1,000) and distance cut-off (500). A p-value cutoff of 0.05 was used to identify peaks unique to each condition. Each resulting peak set was examined for enriched transcription factor binding site motif instances using the HOMER tool suite, modified to use a log base 2 scoring system and include the set of human motifs contained in build 2.0 of the Cis-BP database.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### REFERENCES

Arroyo, J. D., Chevillet, J. R., Kroh, E. M., Ruf, I. K., Pritchard, C. C., Gibson, D. E., Mitchell, P. S., Bennett, C. F., Pogosava-Agadjanyan, E. L., Stirewalt, D. L., et al. (2011). Argonaute2 complexes carry a population of circulating microRNAs independent of vesicles in human plasma. Proc Natl Acad Sci. 108, 5003-5008.

Bhutani, N., Brady, J. J., Damian, M., Sacco, A., Corbel, S. Y., and Blau, H. M. (2010). Reprogramming towards pluripotency requires AID-dependent DNA demethylation. Nature 463, 1042-1047.

Boroviak, T., Stirparo, G. G., Dietmann, S., Hernando-Herraez, I., Mohammed, H., Reik, W., Smith, A., Sasaki, E., Nichols, J., and Bertone, P. (2018). Single cell transcriptome analysis of human, marmoset and mouse embryos reveals common and divergent features of preimplantation development. Development 145.

Caneparo, L., Pantazis, P., Dempsey, W., and Fraser, S. E. J. P. o. 2011). Intercellular bridges in vertebrate gastrulation. 6, e20230.

Collier, A. J., Panula, S. P., Schell, J. P., Chovanec, P., Reyes, A. P., Petropoulos, S., Corcoran, A. E., Walker, R., Douagi, I., and Lanner, F. (2017). Comprehensive cell surface protein profiling identifies specific markers of human naive and primed pluripotent states. Cell Stem Cell 20, 874-890. e877.

Collier, A. J., Rugg-Gunn, P. J. (2018). Identifying human naïve pluripotent stem cells—evaluating state-specific reporter lines and cell-surface markers. BioEssays, 40, 1700239.

Gerdes, H.-H., Rustom, A., and Wang, X. J. M. o. d. (2013), Tunneling nanotubes, an emerging intercellular communication route in development. 130, 381-387.

Guo, G., von Meyenn, F., Rostovskaya, M., Clarke, J., Dietmann, S., Baker, D., Sahakyan, A., Myers, S., Bertone, P., Reik, W., et al. (2017). Epigenetic resetting of human pluripotency. Development 144, 2748-2763.

Gurdon, J. B. (1962). Adult frogs derived from the nuclei of single somatic cells. Dev Biol 4, 256-273.

Haimovich, G., Ecker, C. M., Dunagin, M. C., Eggan, E., Raj, A., Gerst, J. E., and Singer, R. H. PNAS. (2017). Intercellular mRNA trafficking via membrane nanotube-like extensions in mammalian cells. 114, E9873-E9882.

Hou, P., Li, Y., Zhang, X., Liu, C., Guan, J., Li, H., Zhao, T., Ye, J., Yang, W., Liu, K., et al. (2013). Pluripotent stem cells induced from mouse somatic cells by small-molecule compounds. Science 341, 651-6:54.

Karlikow, M., Goic, B., Mongelli, V., Salles, A., Schmitt, C., Bonne, I., Zurzolo, C., and Saleh, M.-C. J. S. r. (2016). Drosophila cells use nanotube-like structures tomtransfer dsRNA and RNAi machinery between cells. 6, 27085.

Kilens, S., Meistermann, D., Moreno, D., Chariau, C., Gaignerie, A., Reignier, A., Lelievre, Y., Casanova, M., Vallot, C., Nedellec, S., et al. (2018a). Parallel derivation of isogenic human primed and naive induced pluripotent stem cells. Nat Commun 9, 360.

Kolodny, G. M. (1971). Evidence for transfer of macromolecular RNA between mammalian cells in culture. Exp Cell Res 65, 313-324.

Kumari, D. (2016). States of pluripotency: naïve and primed pluripotent stem cells, Pluripotent Stem Cells—From the Bench to the Clinic, Minoru Tomizawa. IntechOpen.

Pastor, W. A., Liu, W., Chen, D., Ho, J., Kim, R., Hunt. T. J., Lukianchikov, A., Liu, X., Polo, J. M., and Jacobsen, S. E. J. N. c. b. (2018). TFAP2C regulates transcription in human naive pluripotency by opening enhancers. 20, 553-564.

Pereira, C. F., Terranova, R., Ryan, N. K., Santos, J., Morris, K. J., Cui, W., Merkenschlager, M., and Fisher, A. G. (2008). Heterokaryon-based reprogramming of human B lymphocytes for pluripotency requires Oct4 but not Sox2. PLoS Genet 4, e1000170.

Ramirez-Weber, F. A., and Kornberg, T. B. (1999). Cytonemes: cellular processes that project to the principal signaling center in Drosophila imaginal discs. Cell 97, 599-607.

Rao, R. R., and Stice, S. L. (2004). Gene expression profiling of embryonic stem cells leads to greater understanding of pluripotency and early developmental events. Biol Reprod 71, 1772-1778.

Roy, S., Huang, H., Liu. S., and Kornberg, T. B. (2014). Cytoneme-mediated contact-dependent transport of the Drosophila decapentaplegic signaling protein. Science 343, 1244624.

Salas-Vidal, E., and La eh, H. J. D. b. (2004). Imaging filopodia dynamics in the mouse blastocyst. 265, 75-89.

Sartori-Rupp, A., Cervantes, D. C., Pepe, A., Ciousset, K., Delage, E., Corroyer-Dulmont, S., Schmitt, C., Krijnse-Locker, J., and Zurzolo, C. J. N. c. (2019). Correlative cryo-electron microscopy reveals the structure of TNTs in neuronal cells. Nat Comm. 10(1):342.

Sim, Y.-J., Kim, M,-S., Nayfeh, A., Yun, Y.-J., Kim, S.-J., Park, Kim, C.-H., and Kim, K.-S. J. S. c. r. (2017). 2i Maintains a naive ground state in ESCs through two distinct epigenetic mechanisms. 8, 1312-1328.

Tada, M., Tada, T., Lefebvre, L, Barton, S. C., and Surani, M. A. (1997). Embryonic germ cells induce epigenetic reprogramming of somatic nucleus in hybrid cells. EMBO J 16, 6510-6520.

Takahashi, K., Tanabe, K., Ohnuki, M., Narita, M., Ichisaka, T., Tomoda, K., and Yamanaka, S. (2007). Induction of pluripotent stein cells from adult human fibroblasts by defined factors. Cell 131, 861-872.

Takahashi, K., and Yamanaka, S. (2006). Induction of pluripotent stem cells from mouse embryonic and adult fibroblast cultures by defined factors. Cell 126, 663-676.

Takahashi, S., Kobayashi, S., Hiratani, I. J. C., and sciences, m.l. (2018). Epigenetic differences between naïve and primed pluripotent stem cells. 75, 1191-1203.

Takashima, Y., Guo, G., Loos, R., Nichols, J., Ficz, G., Krueger, F., Oxley, D., Santos, F., Clarke, J., and Mansfield, W. J. C. (2014). Resetting transcription factor control circuitry toward ground-state pluripotency in human. 158, 1254-1269.

Takebe, T., Sekine, K., Enomura, M., Koike, H., Kimura, M., Ogaeri, T., Zhang, R.-R., Ueno, Y., Zheng, Y.-W., and Koike, N. J. N. (2013). Vascularized and functional human liver from an iPSC-derived organ bud transplant. 499, 481.

Tamm, C., Galitó, S. P., and Annerén, C. J. P. o. (2013). A comparative study of protocols for mouse embryonic stem cell culturing. 8, e81156.

Tyml, K., Wang, X., Lidington, D., Ouellette, Y. Am J Physiol Heart Circ Physiol. (2001). Lipopolysaccharide reduces intercellular coupling in vitro and arteriolar conducted response in vivo. 281, H1397-H1406.

Valadi, H., Ekstrom, K., Bossios, A., Sjostrand, M., Lee, J. J., and Lotvall, J. O. (2007). Exosome-mediated transfer of mRNAs and microRNAs is a novel mechanism of genetic exchange between cells. Nat Cell Biol 9, 654-659.

Wakayama, T., Perry, A. C., Zuccotti, M., Johnson, K. R., and Yanagimachi, R. (1998). Full-term development of mice from enucleated oocytes injected with cumulus cell nuclei. Nature 394, 369-374.

Ware, C. B. J. S. C. (2017). Concise review: lessons from naïve human pluripotent cells. 35, 35-41.

Wernig, M., Meissner, A., Foreman, R. Brambrink, T., Ku, M., Hochedlinger, K., Bernstein, B. E., and Jaenisch, R. (2007). In vitro reprogramming of fibroblasts into a pluripotent ES-cell-like state. Nature 448, 318-324.

Wilmut, I., Schnieke, A. E., McWhir, J., Kind, A. J., and Campbell, K. H. (1997). Viable offspring derived from fetal and adult mammalian cells. Nature 385, 810-813.

Zhang, R.-R., Koido, M., Tadokoro, T., Ouchi, R., Matsuno, T., Ueno, Y., Sekine, K., Takebe, T., and Taniguchi, H. J. S. c. r. (2018). Human iPSC-Derived Posterior Gut Progenitors Are Expandable and Capable of Forming Gut and Liver Organoids. 10, 780-793.

Zhao, Y., Zhao, T., Guan, J., Zhang, X., Fu, Y., Ye, J., Zhu, J., Meng, G., Ge, J., Yang, S., et al. (2015). A XEN-like State Bridges Somatic Cells to Pluripotency during Chemical Reprogramming. Cell 163, 1678-1691.

What is claimed is:

1. A method comprising contacting in vitro an acceptor cell with a donor cell, wherein said contacting causes transfer of an intra-cellular component from said donor cell to said acceptor cell.

2. The method of claim 1, wherein said acceptor cell is a pluripotent stem cell (PSC).

3. The method of claim 1 or 2, wherein said acceptor cell is a cell in a primed state expressing primed transcription factors and/or primed cell surface markers.

4. The method of any one of the preceding claims, wherein said acceptor cell is a primed human induced pluripotent stem cell (“primed hiPSC”).

5. The method of any one of the preceding claims, wherein said donor cell comprises tunneling nanotubes (TNTs) or cytonemes on its surface.

6. The method of any one of the preceding claims, wherein said donor cell is a cell in a naïve state expressing naïve transcription factors and/or naïve cell surface markers.

7. The method of any one of the preceding claims, wherein said donor cell is a naïve mouse embryonic stem cell (naïve mESC).

8. The method of any one of the preceding claims, wherein said donor cell and said acceptor cell are contacted in a culture media.

9. The method of any one of the preceding claims, wherein said donor cell and said acceptor cell are cultured in a ratio of at least 20%:80%,

10. The method of any one of the preceding claims, wherein said donor cell and said acceptor cell are cultured in a ratio of 50%:50% or about 50%:50%.

11. The method of any one of the preceding claims, wherein said intra-cellular component is selected from one or more of RNA, protein, and organelles.

12. The method of any one of the preceding claims, wherein said intra-cellular component is RNA.

13. The method of claim 12, wherein the RNA is mRNA, ncRNA, lncRNA, miRNA, piRNA, siRNA, or shRNA.

14. The method of any one of the preceding claims, wherein said donor cell transfers the intra-cellular component via TNTs or cytonemes.

15. The method of any one of the preceding claims, wherein the donor cell and acceptor cell are contacted under hypoxic conditions.

16. The method of claim 15, wherein said hypoxic condition is 5% O₂ or about 5% O₂.

17. The method of any one of claims 1 through 16, wherein the donor cell and acceptor cell are contacted under a stressor condition, wherein said stressor condition is selected from contact with a cell-toxic compound, hypoxia, non-physiological temperature, non-physiological pH, electroporation, or any combination thereof.

18. The method of any one of claims 1 through 17, wherein said cells are contacted or grown at 37° C. or about 37° C.

19. The method of any one of the preceding claims, wherein said acceptor cell and said donor cell are cultured in direct contact.

20. The method of any one of the preceding claims, wherein the donor cell and the acceptor cell are not separated with a transwell.

21. The method of any one of the preceding claims, wherein said contacting step is carried out until said acceptor cell expresses or upregulates expression of naïve stem cell markers comprising one or more of CD130, CD77, CD7, CD75, or F11R.

22. The method of any one of the preceding claims, wherein said contacting step is carried out until said acceptor cell expresses or upregulated expression of naïve stem cell markers comprising one or more of CD130 or CD77.

23. The method of any one of the preceding claims, wherein said contacting step is carried out until said acceptor cell exhibits downregulation of primed stem cell markers comprising one or more of CD90, HLAABC, CD24, CD57 or SSEA4.

24. The method of any one of the preceding claims, wherein said contacting step is carried out until said acceptor cell exhibits downregulation of primed stem cell markers comprising one or more of CD90 or HLA-ABC.

25. The method of any one of the preceding claims, wherein said contacting step is carried out until said acceptor cell expresses or upregulates expression of naïve pluripotency markers comprising one or more of KLF4, KLF5, KLF17, TFCP2L1, DNMT3L, DPPA3, DPPA5, PRDM14, SALL4, ESRRB, TFAP2C, or TBS.

26. The method of any one of the preceding claims, wherein said contacting step is carried out until said acceptor cell expresses or upregulates expression of naïve pluripotency markers comprising one or more of DPPA3, TFCP2L1, DNMT3L, KLF4 and KLF17.

27. The method of any one of the preceding claims, wherein said contacting step is carried out until said acceptor cell exhibits downregulation of primed pluripotency markers comprising one or more of ZIC2, ZIC3, OTX2, DUSP6, FOXA2, or XIST.

28. The method of any one of the preceding claims, wherein said contacting step is carried out until said acceptor cell exhibits downregulation of primed pluripotency markers comprising one or more of DUSP6, THY1.

29. The method of any one of the preceding claims, wherein said contacting step is carried out until the acceptor cell forms dome-shaped naïve acceptor cell colonies.

30. The method of any one of the preceding claims comprising contacting one or both of said donor or acceptor cell with at least one agent selected from the group consisting of resveratrol, epigallocatechin gallate (EGCG), curcumin, genistein, activin-A, Wnt-3a, sodium butylate, basic fibroblast growth factor (bFGF), oncostatin M (OSM), dexamethasone (DEX), hepatocyte growth factor (HGF), CHIR-99021, forskolin, Y-27632 (ROCK inhibitor), (s)-(−)-blebbistatin, IWP2, A83-01, LY294002, SB-431542, NVP-BHG, Cyclopamine-KAAD, PD-0325901, FGF4, LDN-193189, insulin like growth factor (IGF), bone morphogenetic protein 2 (BMP2), transforming growth factor β2 (TGF-β2), BMP4, FGF-7, platelet-derived growth factor (PDGF) β3, epidermal growth factor (EGF), exendin-4, human neuregulin (PIRG) β3, retinoic acid (RA), L-Ascorbic acid 2-phosphate (AA2P), ascorbic acid, insulin-transferrin-selenium ethanolamine solution (ITS-X), insulin, rifampicin, penicillin, streptomycin, 2-mercaptoethanol, 3-mercaptopropane-1,2-diol (thioglycerol), L-proline, L-glutamine, non-essential amino acid mixture (NEAA), sodium pyruvate, trypsin-EDTA, phosphatidylinositol (PI), interleukins, prostaglandins, and tumor necrosis factors, or any combination thereof.

31. The method of any one of the preceding claims, wherein the donor cell and the acceptor cell are cultured for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days.

32. The method of any one of the preceding claims, wherein the donor cell and the acceptor cell are cultured in naïve maintenance medium.

33. The method of claim 32, wherein the naïve maintenance medium is PXGL medium, N2B27 medium, N2 medium, tt2iLGö medium, or 2i medium with gelatin.

34. The method of any one of the preceding claims, wherein the donor cell and the acceptor cell are not contacted or cultured with an HDAC inhibitor.

35. The method of any one of claims 1-34, wherein after contacting, the acceptor cell comprises exogenous mRNA from the donor cell that is xenogeneic.

36. The method of claim 35, wherein the acceptor cell expresses protein from the exogenous mRNA from the donor cell that is xenogeneic.

37. The method of any one of claims 1-34, wherein after contacting, the acceptor cell comprises exogenous mRNA from the donor cell that is allogeneic or autologous.

38. The method of claim 37, wherein the acceptor cell expresses protein from the exogenous mRNA from the donor cell that is allogeneic or autologous.

39. The method of any one of the preceding claims, wherein after contacting, the acceptor cell comprises exogenous mRNA encoding for at least 6,392 genes from the donor cell.

40. The method of any one of the preceding claims, wherein after contacting, the acceptor cell comprises exogenous mRNA encoding for at least 491 naïve transcription factors from the donor cell.

11. The method of any one of the preceding claims, wherein the acceptor cell is not modified by transfection, electroporation, or transduction with a virus, or any combination thereof, prior to contacting.

42. The method of any one of the preceding claims, wherein after contacting, the acceptor cell undergoes changes in chromatin accessibility.

43. The method of claim 42, wherein the changes in chromatin accessibility comprises increased accessibility to binding motifs for SOX2, or TFAP2C, or both.

44. The method of any one of the preceding claims, wherein the acceptor cell and donor cell are contacted in naïve stem cell media or naïve maintenance media.

45. A cell composition comprising an acceptor cell and a donor cell, wherein the acceptor cell pluripotent stem cell, and the donor cell is a naïve pluripotent stem cell.

46. The cell composition of claim 45, further comprising a naïve stem cell medium or naïve maintenance medium.

47. The cell composition of claim 45, wherein the naïve stem cell medium or naïve maintenance medium is PXGL medium, N2B27 medium, N2 medium, tt2iLGö medium, or 2i medium with gelatin.

48. The cell composition of any one of claims 45-47, wherein the acceptor cell is a primed pluripotent stem cell.

49. The cell composition of any one of claims 45-48, wherein the acceptor cell is a human primed pluripotent stem cell.

50. The cell composition of any one of claims 45-49, wherein the acceptor cell is a human naïve pluripotent stem cell.

51. The cell composition of any one of claims 45-50, wherein the donor cell is a mouse naïve pluripotent stem cell.

52. The cell composition of any one of claims 45-51, wherein the donor cell is a mouse embryonic stem cell.

53. The cell composition of any one of claims 45-52, wherein the donor cell and the acceptor cell are at a ratio of at least 20%:80%.

54. The cell composition of any one of claims 45-53, wherein the donor cell and the acceptor cell are at a ratio of 50%:50%; or about 50%:50%.

55. The cell composition of any one of claims 45-54, wherein the acceptor cell expresses naïve stem cell markers comprising one or more of CD130, CD77, CD7, CD75, or F11R.

56. The cell composition of any one of claims 45-55, wherein the acceptor cell expresses naïve stem cell markers comprising one or more of CD130 or CD77.

57. The cell composition of any one of claims 45-56, wherein the acceptor cell exhibits downregulation of primed stem cell markers comprising one or more of CD90, HLAABC, CD24, CD57 or SSEA4.

58. The cell composition of any one of claims 45-57, wherein the acceptor cell exhibits downregulation of primed stem cell markers comprising one or more of CD90 or HLA-ABC.

59. The cell composition of any one of claims 45-58, wherein the acceptor cell expresses naïve pluripotency markers comprising one or more of KLF4, KLF5, KLF17, TFCP2L1, DNMT3L, DPPA3, DPPA5, PRDM14, SALL4, ESRRB, TFAP2C, or TBS.

60. The cell composition of any one of claims 45-59, wherein the acceptor cell expresses naïve pluripotency markers comprising one or more of DPPA3, TFCP2L1, DNMT3L, KLF4 and KLF17.

61. The cell composition of any one of claims 45-60, wherein the acceptor cell exhibits downregulation of primed pluripotency markers comprising one or more of ZIC2, ZIC3, OTX2, DUSP6, FOXA2, or XIST.

62. The cell composition of any one of claims 45-61, wherein the acceptor cell exhibits downregulation of primed pluripotency markers comprising one or more of DUSP6, THY1.

63. The cell composition of any one of claims 45-62, wherein the acceptor comprises exogenous mRNA from the donor cell that is xenogeneic.

64. The cell composition of any one of claims 45-62, wherein the acceptor cell comprises exogenous mRNA from the donor cell that is allogeneic or autologous.

65. The cell composition of any one of claims 45-64, wherein the acceptor cell comprises exogenous mRNA encoding for at least 6,392 genes from the donor cell.

66. The cell composition of any one of claims 45-65, wherein the acceptor cell comprises exogenous mRNA encoding for at least 491 naïve transcription factors from the donor cell.


SEQUENCE LISTING (18 sequences)

SEQ ID NO: 1 - DNA, 21 bp, Artificial Sequence
  mouse Klf4 shRNA #1
gcaagtcagcttgtgaatgga

SEQ ID NO: 2 - DNA, 22 bp, Homo sapiens
actgagtcatcttgtgagtgga

SEQ ID NO: 3 - DNA, 21 bp, Artificial Sequence
  Mouse Klf4 shRNA #2
gcaatacacacgtaaagatca

SEQ ID NO: 4 - DNA, 22 bp, Homo sapiens
gcaatataagcataaaagatca

SEQ ID NO: 5 - DNA, 22 bp, Artificial Sequence
  Mouse Klf4 shRNA #3
tgttctaacagcctaaatgatg

SEQ ID NO: 6 - DNA, 22 bp, Homo sapiens
tgtttaaatagcctaaatgatg

SEQ ID NO: 7 - DNA, 21 bp, Artificial Sequence
  Mouse Tfcp2l1 shRNA #1
ggattatggtggatattatgg

SEQ ID NO: 8 - DNA, 21 bp, Homo sapiens
gaggattaggaatgagtattc

SEQ ID NO: 9 - DNA, 21 bp, Artificial Sequence
  Mouse Tfcp2l1 shRNA #2
gaaacaattgtcatcattatt

SEQ ID NO: 10 - DNA, 21 bp, Homo sapiens
cttgctaaaggcctctgcttc

SEQ ID NO: 11 - DNA, 21 bp, Artificial Sequence
  Tfcp2l1 shRNA #3
gccatgaaatgctagggtagg

SEQ ID NO: 12 - DNA, 21 bp, Homo sapiens
cgtacaaaaagctggacgtgg

SEQ ID NO: 13 - DNA, 21 bp, Artificial Sequence
  Mouse Tfap2c shRNA #1
gcatgggagtcgtcagataaa

SEQ ID NO: 14 - DNA, 10 bp, Homo sapiens
tgatgtgccc

SEQ ID NO: 15 - DNA, 21 bp, Artificial Sequence
  Mouse Tfap2c shRNA #2
gatctgtaaatacttcttatg

SEQ ID NO: 16 - DNA, 29 bp, Homo sapiens
ggtctgtaaatactggtgcacttcttacg

SEQ ID NO: 17 - DNA, 21 bp, Artificial Sequence
  Mouse Tfap2c shRNA #3
gtattgaaactgaagatgaag

SEQ ID NO: 18 - DNA, 21 bp, Homo sapiens
gtattaagaattaagaggatg